(19) United States
(12) Patent Application Publication
(10) Pub. No.: US 2025/0312408 A1
(43) Pub. Date: Oct. 9, 2025

(54) NAP FOR SEX-SPECIFIC TREATMENT OF DISEASES

(71) Applicant: RAMOT AT TEL-AVIV UNIVERSITY LTD., Tel Aviv (IL)

(72) Inventors: Illana GOZES, Tel Aviv (IL); Guy SHAPIRA, Tel Aviv (IL); Alexandra LOBYNTSEVA, Tel Aviv (IL); Gidon KARMON, Tel Aviv (IL); Noam SHOMRON, Tel Aviv (IL)

(21) Appl. No.: 18/718,925

(22) PCT Filed: Dec. 13, 2022
(86) PCT No.: PCT/IL2022/051314

§ 371 Date: Jun. 12, 2024

Related U.S. Application Data
(60) Provisional application No. 63/289,174, filed on Dec. 14, 2021.
(60) Provisional application No. 63/306,146, filed on Feb. 3, 2022.

(51) Int. Cl. A61K 38/08; A61P 25/18; A61P 25/28

(52) U.S. Cl. CPC A61K 38/08; A61P 25/18; A61P 25/28

(57) ABSTRACT

The present invention provides methods for to sex-specific treatment and dose titration of diseases associated with an aberrant functionality of activity-dependent neuroprotective protein (ADNP) and/or cytoskeleton such as progressive supranuclear palsy (PSP), schizophrenia, amnestic mild cognitive impairment (aMCI), Alzheimer's disease, and autism. The treatment in these diseases, e.g., the dose and/or the regimen, differs between sexes and has to be adapted to obtain the desired effect. Specifically, use of davunetide in treatment of women suffering from PSP or of men suffering from schizophrenia or aMCI are provided.

### SEQUENCE LISTING

The instant application contains a Sequence Listing which has been submitted electronically in xml format and is hereby incorporated by reference in its entirety. Said xml file, created on 14 Nov. 2024, is named RAMOT-0116 PCT-revised14.11.24.xml and is 64,782 bytes in size.

### FIELD OF THE INVENTION

The present invention relates to sex-specific treatments of diseases associated with an aberrant functionality of activity-dependent neuroprotective protein (ADNP) or tightly linked with microtubule (cytoskeletal) function, such as the treatment of females suffering from progressive supranuclear palsy, males suffering from schizophrenia or amnestic mild cognitive impairment.

### BACKGROUND OF THE INVENTION

NAP (NAPVSIPQ single amino acid letter code, also known as davunetide, AL-108, CP201 and sometimes referred to as NAP peptide) is the smallest neuroprotective peptide site of activity-dependent neuroprotective protein (ADNP). NAP and pipeline products protect nerve cells by associating with microtubule end binding proteins (EB1/EB3), through the SxIP motif (NAPVSIPQ), thus enhancing microtubule dynamics and Tau-microtubule interaction, protecting the synapse. NAP further enhances ADNP-EB1/EB3, interactions, protecting against ADNP deficits (Hacohen-Kleiman, et al., The Journal of clinical investigation 2018; 128(11): 4956-4969). Recently, a homology 3 (SH3) domain-ligand association site in NAP (NAPVISP) responsible for controlling signaling pathways regulating the cytoskeleton, including actin-Tau interactions was shown (Ivashko-Pachima et al., Mol Psychiatry. 2022; 27(8):3316-3327).

Progressive supranuclear palsy (PSP) is a rare and fatal neurodegenerative movement disorder and no disease modifying therapy (DMT) is currently available. Symptoms include changes to speech, balance, walking, swallowing, vision, cognition, autonomic functioning as well as Parkinson's like symptoms, such as, tremor, stiffness and slowness. PSP is a rapidly progressive, neurodegenerative disease, a tauopathy caused by abnormal folding of the protein Tau in brain cells.

The most prevalent tauopathy is Alzheimer's disease while other less frequent tauopathies exist as well. Tauopathy can also be found in other brain diseases, including, but not limited to amyotrophic lateral sclerosis (ALS) and autism. All these diseases are currently lacking disease modifying therapeutics.

More encompassing, progressive supranuclear palsy (PSP), corticobasal degeneration (CBD) and multiple system atrophy (MSA) are brain diseases that can cause changes to speech, balance, walking, swallowing, vision, cognition and autonomic functioning. These diseases can also involve symptoms similar to Parkinson's disease including tremor, stiffness and slowness. Because of this, people may initially be diagnosed with Parkinson's and you may hear PSP, CBD and MSA referred to as “atypical parkinsonism” syndromes. They are neurodegenerative, meaning that symptoms may change and worsen over time.

PSP, CBD and MSA are considered rare, especially compared to the more common neurodegenerative diseases such as Alzheimer's disease and Parkinson's disease. However, only about 25% of people with PSP, CBD and MSA are accurately diagnosed.

Davunetide (NAP) treatment has shown cognitive score enhancement in amnestic mild cognitive (aMCI) (Morimoto et al. Dementia and geriatric cognitive disorders 2013; 35(5-6): 325-336) and protection of daily living activities in schizophrenia patients (Javitt et al., Schizophrenia research 2012; 136(1-3): 25-31; Gozes I. Frontiers in neurology 2020; 11: 608444). The choice of aMCI as a therapeutic target for davunetide is obvious as it protects against tauopathy and cell death, with aMCI being the precursor of Alzheimer's disease, the major tauopathy. The choice of schizophrenia is equally relevant, as by enhancing microtubule dynamics, davunetide augments the autophagy process, which is deficient in schizophrenia. Importantly, ADNP regulates the autophagy process, interacting with the autophagy-controlling proteins, e.g. microtubule-associated protein 1 light chain 3 (LC3) and regulating beclin 1 expression, with NAP (davunetide) enhancing/replacing deficient ADNP deficits and providing neuroprotection (Sragovich S, Merenlender-Wagner A, Gozes I. Bioessays. 2017; 39(11)).

Given that tauopathy is a major outcome of ADNP-deficiency/pathological mutations in mice and humans (Grigg et al., Translational psychiatry 2020; 10(1): 228) and NAP (davunetide) shows protection against tauopathy in multiple preclinical models as well as indicative of increasing cognitive scores in the aMCI trial cited above, a clinical study in a pure tauopathy was carried out in progressive supranuclear palsy (PSP) as an ideal target for davunetide protection. However, the trial, while showing safety, did not meet its endpoints (Boxer et al., The Lancet Neurology 2014; 13(7): 676-685).

In trying to understand the results, one explanation suggested a dose beyond a bell-shape dose response, identified in preclinical studies (Bassan et al., Journal of neurochemistry 1999; 72(3): 1283-1293; Leker et al., Stroke 2002; 33(4): 1085-1092) and further suggested in clinical trials. Thus, in schizophrenia, a 5 mg daily dose was significantly more effective than placebo while a higher 15 mg twice daily was insignificantly different from the 5 mg daily dose as well as the placebo, possibly due to under-powering and high variability, however, suggestive of plateauing toward a bell shape response (Javitt et al.). In aMCI, there was a dose dependence (with the above doses), suggesting disease-specificity (Morimoto et al). However, the dose used in PSP (30 mg, twice daily) was twice higher than the highest aMCI and schizophrenia doses possibly reaching a bell shape response.

Another search for an explanation for the trial results looked more closely at Tau. Indeed, understanding the regulation of the Tau (MAPT) mRNA splicing is important for the purpose of solving the etiology of PSP as well as other tauopathies. Tauopathies can be categorized by the presence of tau aggregates containing 3 (3R) and/or 4 (4R) microtubule-binding domain repeats (determined by inclusion/exclusion of MAPT exon 10) and by inclusion/exclusion of exons 2 and 3 translated to two N-terminal Tau domains, with the accumulation of 1N4R isoforms in PSP and 0N isoforms in the Alzheimer's disease temporal cortex. Importantly, NAP (davunetide)/ADNP enhances Tau microtubule association (Ivashko-Pachima et al., Molecular psychiatry 2017; 22(9): 1335-1344) in turn, ADNP is directly interacting with the splicing machinery, possibly to suppress exon 10 inclusion. Furthermore, while the davunetide trial in the pure 4-repeat Tau, PSP, a potentially ideal population target for davunetide protection, was deemed negative (Boxer et al.,), retrospectively, part of the negative result was attributed to NAP preferential enhancement of the dynamic 3-repeat Tau (containing 3 microtubule interaction sites) vs. the 4-repeat Tau microtubule interaction (Ivashko-Pachima et al., PLoS One 2019; 4(3)):e0213666).

Lastly, in trying to understand what drives PSP tauopathy, an elegant study identified genetic variations and increases in filamin-A in the PSP brains. As such, increased filamin-A levels enhanced the phosphorylation and insolubility of tau through interacting actin filaments. In addition, the reduction of filamin-A corrected aberrant tau levels in the culture cells from PSP cases and transgenic mice carrying human filamin-A recapitulated tau pathology in the neurons (Tsujikawa et al., Science advances 2022; 8(21): eabm5029). In this respect, we have shown that ADNP contains an actin binding domain and NAP corrects actin-associated protein interactions (Ivashko-Pachima et al., Molecular psychiatry 2022; 27(8):3316-3327).

The effect of NAP was tested in patients suffering schizophrenia, yet another neurological disease associated with aberrant ADNP (Javitt et al., Schizophrenia research 2012; 136(1-3): 25-31. The score of UCSD Performance-based Skills Assessment (UPSA) after administration of subjects treated with a 5 mg daily dose was significantly higher than in placebo group. The score of patients treated with a higher dose of 30 mg (15 mg twice daily) was insignificantly different from the 5 mg daily dose as well as the placebo.

PSP and other neurodegenerative diseases still have no treatment and there is an urgent need for providing such. Similarly, cognitive impairments in schizophrenia are not adequately addressed and developmental disorders, including, but not limited to, autism spectrum disorders, Alzheimer's disease are an unmet medical need.

### SUMMARY OF THE INVENTION

The present invention is based on an unexpected observation that males and females suffering from PSP react differently to the treatment by NAP peptide (also called davunetide).

We show in the Examples that while sick males did not benefit from the therapy by NAP, a significant improvement was seen in treated women. We found that NAP peptide reduced the increase in the size of brain ventricles caused by neurodegeneration in females. This was not observed in males. Finding that NAP protein may be used in treating PSP contradicts the previous statements of e.g., Boxer et al., (The Lancet Neurology 2014; 13(7): 676-685) explicitly stating that Davunetide is not an effective treatment for PSP. In addition, based on our understanding of the mechanism of tauopathies associated with aberrant ADNP protein and specifically the development of Alzheimer's disease we find that this sex-specific treatment is effective in these diseases and conditions. Further, as the neurodegenerative side effect of several biological anti-amyloid-β therapies, e.g. of aducanumab, is expressed in the increase of brain ventricles, we find that NAP may be beneficial in reducing said side effect, specifically in a sex-specific mode. Further, we showed that the treatment with NAP 30 mg/day of males suffering from schizophrenia provides a significant improvement in their symptoms. This was not observed for females. A similar observation was made for males suffering from amnestic mild cognitive impairment; the treatment with NAP 30 mg/day of males suffering provided a significant improvement in their symptoms. It seems that much higher doses are required for females. Thus, according to one aspect, the present invention provides a pharmaceutical composition comprising a peptide comprising the amino acid sequence SEQ ID NO: 1 (NAPVSIPQ; NAP peptide) and a pharmaceutically acceptable carrier, for use in a sex-specific treatment or inhibition of the development of a disease or disorder associated with an aberrant functionality of activity-dependent neuroprotective protein (ADNP) in a subject. According to some embodiments, the disease or disorder is selected from a neurodegenerative disease, neurodevelopmental disease or mental disease, and tauopathy. According to some embodiments, the disease is taupathy. According to some embodiments, the disease or disorder is selected from progressive supranuclear palsy (PSP), schizophrenia, Alzheimer's disease, amnestic mild cognitive impairment (aMCI), ADNP syndrome, autism and muscle disease.

According to some embodiments, the disease is progressive supranuclear palsy (PSP) and the use comprises treating or inhibiting the development of PSP in a female subject. In some embodiments, the treatment or prevention of the development of PSP comprises preventing or inhibiting the increase in the size of brain ventricles. According to some embodiments, the use comprises administering from 1 to 80 mg/day of the NAP peptide. According to some embodiments, the use comprises administering from 40 to 80 mg/day of the NAP peptide.

According to some embodiments, the disease is progressive supranuclear palsy (PSP) and use comprises treating PSP in a male subject comprising administering to said subject from 1 to 50 mg/day of the NAP

According to some embodiments, the disease is schizophrenia and the use comprises treating schizophrenia in a male subject, wherein the use comprises administering to said male subject from 10 to 80 mg/day of the NAP peptide. According to some embodiments, the use comprises administering to said male subject from 20 to 80 mg/day of the NAP peptide.

According to some embodiments, the disease is schizophrenia and the use comprises treating schizophrenia in a female subject, wherein the use comprises administering to said subject from 1 to 4 mg/day of the NAP peptide.

According to some embodiments, the disease is amnestic mild cognitive impairment (aMCI) and the use comprises treating amnestic aMCI in a male subject, wherein the use comprises administering to said subject from 10 to 80 mg/day or from 25 to 80 mg/day of the NAP peptide.

According to some embodiments, the disease is amnestic mild cognitive impairment (aMCI) and the use comprises treating amnestic aMCI in a female subject, wherein the use comprises administering to said subject from 35 to 80 mg/day of the NAP peptide.

According to some embodiments, the use comprises sex-specific treatment or prevention of Alzheimer's disease. According to some embodiments, the use comprises treatment or prevention at the prodromal stage of the disease. According to some embodiments, Alzheimer's disease is characterized by the presence in a biological sample of at least one of the followings: (i) of aberrant P53 protein; (ii) P-tau217; (iii) P-tau231 and (iii) P-tau181 and (iv) change in ADNP expression.

According to some embodiments, the use comprises treating a female subject and comprises administering from 1 to 80 mg/day, from 1 to 10 mg/day, from 20 to 40 mg/day or from 40 to 80 mg/day of the NAP.

According to some embodiments, the use comprises treating a male subject and comprises administering from 1 to 80 mg/day, from 1 to 10 mg/day, from 20 to 40 mg/day or from 40 to 80 mg/day of the NAP.

According to another aspect, the present invention provides a method for treating or inhibiting the development of a disease or disorder associated with an aberrant functionality of activity-dependent neuroprotective protein (ADNP) in a subject comprising administering to said subject a peptide comprising the amino acid sequence SEQ ID NO: 1 (NAPVSIPQ; NAP peptide) in a sex-specific way/manner.

According to some embodiments, the present invention provides a method for treating or inhibiting the development of PSP in a female subject comprising administering to said female subject a peptide comprising the amino acid sequence SEQ ID NO: 1. According to some embodiments, the method comprises administering from 40 to 80 mg/day of the NAP peptide.

According to some embodiments, the present invention provides a method for treating or inhibiting the development of PSP in a male subject comprising administering to said subject from 1 to 50 mg/day of the NAP

According to some embodiments, the present invention provides a method for treating schizophrenia in a male subject, comprising administering to said male subject from 10 to 80 mg/day of the NAP peptide. According to some embodiments, the method comprises administering to said male subject from 20 to 80 mg/day of the NAP peptide.

According to some embodiments, the present invention provides a method for treating schizophrenia in a female subject, wherein the use comprises administering to said subject from 1 to 4 mg/day of the NAP peptide.

According to some embodiments, the present invention provides a method for treating amnestic mild cognitive impairment (aMCI) comprising treating amnestic aMCI in a male subject, wherein the use comprises administering to said subject from 10 to 80 mg/day or from 25 to 80 mg/day of the NAP peptide.

According to some embodiments, the present invention provides a method for treating amnestic aMCI in a female subject, wherein the use comprises administering to said subject from 35 to 80 mg/day of the NAP peptide.

According to some embodiments, the present invention provides a method for treating or preventing of Alzheimer's disease in a subject in need thereof in sex-specific manner. According to some embodiments, treatment or prevention is at the prodromal stage of the disease.

According to some embodiments, Alzheimer's disease is characterized by the presence in a biological sample of at least one of the followings: (i) of aberrant P53 protein; (ii) P-tau217; (iii) P-tau231 and (iii) P-tau181 and (iv) change in ADNP expression. According to some embodiments, the method comprises treating a female subject and comprises administering from 1 to 80 mg/day, from 1 to 10 mg/day, from 20 to 40 mg/day or from 40 to 80 mg/day of the NAP. According to some embodiments, the method comprises treating a male subject and comprises administering from 1 to 80 mg/day, from 1 to 10 mg/day, from 20 to 40 mg/day or from 40 to 80 mg/day of the NAP.

According to another aspect, the present invention provides a pharmaceutical composition comprising a peptide comprising the amino acid sequence NAPVSIPQ and a pharmaceutically acceptable carrier for use in ameliorating and/or preventing side effects of an anti-amyloid-β therapy, wherein the therapy comprises anti-amyloid-β antibodies. According to some embodiments, the composition is administered in a sex-specific mode.

According to some embodiments, the composition is administered to a female subject, e.g., in a dose of from 1 to 70 mg/day or from 50 to 70 mg/day of the NAP. 1351 According to some embodiments, the composition is administered to a male subject, e.g. in a dose of from 1 to 70, from 1 to 50 or from 50 to 70 mg/day of the NAP.

According to some embodiments, the anti-amyloid-β therapy comprises aducanumab or lecanemab.

According to any one of the above aspects and embodiments, the NAP peptide or the pharmaceutical composition comprising NAP peptide is administered intranasally.

According to any one of the above aspects and embodiments the sex-specific treatment comprises inhibiting neurodegeneration in the subject.

### BRIEF DESCRIPTION OF DRAWINGS

FIG. 1 shows baseline demographics (FIG. 1A—weight; FIG. 1B—height; FIG. 1C—age; FIG. 1D—brain ventricle volume; FIG. 1E—SEADL; FIG. 1F—PSPRS) for PSP—suffering subjects treated with placebo or NAP and divided by sex: for that at the baseline, the tested population differed by weight, height and ventricular volumes, with obvious higher male values.

FIG. 2 shows linear regressions separating males and females, davunetide and placebo-treated as measured by MRI, percent of baseline change in ventricular volume at 52 weeks of treatment, correlating with age.

FIG. 3 shows linear regressions separating males and females, davunetide and placebo-treated as measured by MRI, change in ventricular volume at 52 weeks of treatment, correlating with ventricular baseline ventricular volume (mm).

FIG. 4 shows total Tau (FIG. 4A) and phosphorylated Tau (FIG. 4B) reduction in the cerebrospinal fluid (CSF) of the davunetide-treated female patients compared to placebo.

FIG. 5 shows correlations plot r values employing percent change compared to baseline at 52 week davunetide vs. placebo treatment results including the study primary endpoints, PSPRS and SEADL, the secondary end point of brain ventricular volume change as well as age. Significance is denoted by: *P<0.05; **P<0.01; ***P<0.001. “.” denotes #P<0.1 and >0.05.

FIG. 6 shows linear regression plots comparing, the male and female placebo groups change over the 52 weeks trial (FIG. 6A) PSPRS, (FIG. 6B) limb motor domain of the PSPRS, (FIG. 6C) SEADL, (FIG. 6D) table showing statistically significant accelerated deterioration in the davunetide-treated males. *indicates significance and #P<0.1 and >0.05, compared to placebo as detailed on the figure.

FIG. 7 shows linear regression plots comparing, female davunetide vs. placebo change over the 52 week trial (FIG. 7A) PSPRS, (FIG. 7B) behavior domain of the PSPRS, (FIG. 7C) bulbar domain of the PSPRS, (FIG. 7D) SEADL. *indicates significance compared to placebo, as detailed on the figure.

FIG. 8 shows the exploratory endpoint GDS change from baseline to 52 weeks of davunetide vs. placebo treatment results in females (FIG. 8A) and males (FIG. 8B).

FIG. 9 shows the effect of davunetide vs. placebo treatment on the change in elderly patients suffering from amnestic mild cognitive impairment (aMCI), on the CANTAB DMTS scale from baseline to 16 weeks. The DMTS scale measured the change in percentage right recognition of the object after 12-second delay (short-term visual and verbal working memory). Patients were treated with two doses of davunetide: 5 mg/day and 30 mg/day (15 mg/bid) over the 12-week trial with every 4-week assessments including an additional assessment 4 weeks after cessation of treatment. The change in the DMTS scale from baseline was measured and presented as follows: FIG. 9A—women, 5 mg/day; FIG. 9B—women, 15 mg/twice daily; FIG. 9C men, 5 mg/day, and FIG. 9D—men, 15 mg/twice daily. **P<0.01, #P<0.1>0.5.

FIG. 10 shows the effect davunetide vs. placebo treatment on the change in UPSA scale from week 1 in patients diagnosed as suffering from schizophrenia. Patients were treated with two doses of davunetide: 5 mg/day and 30 mg/day (15 mg/bid) over the 6-week trial or the 12 week trial. The change in the UPSA scale from week 1 was measured and presented as follows: FIG. 10A—women, 5 mg/day; FIG. 10B—women, 15 mg/twice daily; FIG. 10C men, 5 mg/day, and FIG. 10D—men, 15 mg/twice daily. * indicates significance, as detailed on the figure. # indicates trending. There is a significant difference with P=0.0276 between women and men treatment using 5 mg/day. All statistical comparisons included Student t-tests between two groups.

FIG. 11 shows opposite gene regulation in male and female mice with ADNP syndrome. A complete RNA sequencing of brain hippocampal samples shows an opposite regulation in ADNP mutated HTR males and HTR females compared to control wild type (WT), treated with vehicle (saline) or NAP as depicted for the Hace1 gene/mRNA intron retention event (aberrant alternative splicing) in males, corrected by NAP.

### DETAILED DESCRIPTION OF THE INVENTION

Unless otherwise defined, all technical and/or scientific terms used herein have the same meaning as commonly understood by one of ordinary skill in the art to which the invention pertains. In case of conflict, the patent specification, including definitions, will control.

According to one aspect, the present invention provides a pharmaceutical composition comprising a peptide comprising the amino acid sequence NAP peptide and a pharmaceutically acceptable carrier for use in a sex-specific treatment, inhibition of the development or prevention of a disease or disorder associated with an aberrant functionality of activity-dependent neuroprotective protein (ADNP) in a subject.

According to some embodiments, the disease or disorder is selected from a neurodegenerative disease, neurodevelopmental disease or mental disease, and tauopathy According to some embodiments, the wherein the disease or disorder is a tauopathy.

The term “tauopathy” refers to neurodegenerative disorders characterized by the deposition of abnormal tau protein in the brain. Tauopathy is a major outcome of ADNP-deficiency or ADNP aberrant functionality.

The term aberrant functionality of activity-dependent neuroprotective protein (ADNP) encompasses any deviation from a normal functionally of ADNP protein. This is caused typically by mutations resulted in non-functional, malfunctioning or shortened version of the protein e.g., due to the presence of proteolytic sites.

The term “activity-dependent neuroprotective protein” (ADNP) refers to a human protein factor (Bassan et al.; Zamostiano et al., J Biol Chem. 2001; 276(1):708-14). The protein has neurotrophic/neuroprotective activity as measured e.g., with in vitro cortical neuron culture assays described by, e.g., Gozes et al., (Proc. Natl. Acad. Sci. USA 93, 427-432, 1996; Bassan et al.) and reviewed in Gozes. Book Chapter 13, in Neuroprotection in Alzheimer's Disease, 1st Edition—Dec. 30, 2016, Gozes, Ed., Academic Press). ADNP regulates hundreds of genes. Non-limiting example is Hace1 as shown in Example 5. Hace1 (HECT Domain And Ankyrin Repeat Containing E3 Ubiquitin Protein Ligase 1) associated with ataxia (Bellamy et al., PLoS One. 2022; 17(1):e0261845), Huntington disease (Ehrnhoefer et al., Hum Mol Genet. 2018; 27(2):239-253. With mutations resulting severe neurodevelopmental disorder (OMIM: 616756), and with further involvement in depression (Ciuculete et al., Clin Epigenetics. 2020; 12(1):99) and Alzheimer's disease (Ni et al., J Alzheimers Dis. 2018; 64(4):1149-1161).

The terms “NAP” and “NAP protein” are used herein interchangeably and refer to an 8-amino acid peptide consisting of amino acids NAPVSIPQ set forth in SEQ ID NO: 1 and known also as davunetide (AL-108, also known as CP201).

Nevertheless, in its broadest definition, the term “NAP peptide” refers to peptides comprising the amino acid sequence NAPVSIPQ and to any derivative or analog of the peptide comprising the amino acids NAPVSIPQ or related sequences and having the same biological activity, e.g., as peptides defined in WO2008084483, WO2006099739, US2012208763 and US20150141345 and incorporated herein by reference in their entirety. According to some embodiments, the term NAP refers to one of the NAP's derivatives having an amino acid sequence selected from amino acid sequences SEQ ID NO: 2-49 and to NAP alpha-aminoisobutyric acid analog, or SKIP, (Ivashko-Pachima et al., J Mol Neurosci. 2021 August; 71(8):1515-1524) or Ac-SKIP (Ivashko-Pachima and Gozes (Front Cell Neurosci. 2019 Oct. 1; 13:435. doi: 10.3389/fncel.2019.00435). According to some embodiments, the methods of treatment of the present invention include use of other compounds that have an activity that is similar to that of NAP, i.e. alternatives of NAP, regulating ADNP. Examples of such NAP's alternatives, regulating ADNP are vasoactive intestinal peptide (VIP) and pituitary adenylate cyclase-activating polypeptide (PACAP) described e.g. in Sragovich et al (Translational Psychiatry volume 9, Article number: 235 (2019); SNV described in Eger et al., (Front Pharmacol. 2021 May 5; 12:638128. doi: 10.3389/fphar.2021.638128), or ketamine (Brown et al., Neuroscience. 2015 Apr. 2; 290:31-40). Furthermore, NAP activity should also be considered in combination with other drugs such as anti-psychotic drugs including but not limited to risperidone or clozapine.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s). In one embodiment, the term treating refers to inhibiting the progression of the disease or disorder. In other embodiments, the term refers to reversing and diminishing the course of progression of the disease or disorder. According to some embodiments, the term refers also to preventing the disease. As used herein, the term “preventing” when used in relation to a condition, refers to the administration of a composition which reduces the frequency of, or delays the onset of, symptoms of a medical condition in a subject relative to a subject who does not receive the composition.

As described above, the main essence of the present invention is that the treatment of the diseases described herein is sex-specific. The term “sex-specific treatment” has the meaning that the treatment of males and females is different in the terms of doses, regime, and outcome. As such, the treatment may be effective to one sex and ineffective to the opposite sex, the dose that is effective to one sex is ineffective or even harmful to the opposite sex etc. The terms “sex-specific” and “gender-specific” may be used interchangeably. The term “sex” refers to the biologic sex of the subject defined by genetics as well known in the art. The terms “male”, “males”, “man” and “men” are used herein interchangeably. The terms “female”, “females”, “woman” and “women” are used herein interchangeably.

The term “pharmaceutical composition” as used herein refers to a composition comprising at least one active agent as disclosed herein optionally formulated together with one or more pharmaceutically acceptable carriers. Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffins.

The pharmaceutical composition of the present invention may be administered in any known method. The term “administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both a direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the administration of the pharmaceutical composition of the present invention is intranasal.

As defined herein above, the disease or disordered treated in a sex-specific manner is a disease or disorder associated with an aberrant functionality of ADNP. Non-limiting examples of such diseases are progressive supranuclear palsy (PSP) and related diseases (CBD, MSA), schizophrenia and related mental disorders including depression, Alzheimer's disease, amnestic mild cognitive impairment (aMCI), Parkinson's disease, neurodevelopmental disorders, including but not limited to, ADNP syndrome, Phelan McDermid syndrome, Dravet syndrome, Rett syndrome, Angelman syndrome, idiopathic or genetic autism spectrum disorder, Down syndrome, amyotrophic lateral disorder (ALS), ataxia, Huntington disease and muscle disease associated with aberrant functionality of ADNP, including, but not limited to natural aging, Duchene muscular dystrophy and Pompe disease. All these diseases or disorders are associated with or mediated by aberrant functionality of ADNP/cytoskeleton, such as aberrant expression, missense, non-sense and frameshift mutations resulting in aberrant length or structure of ADNP.

### PSP

According to one embodiment, the disease is progressive supranuclear palsy (PSP).

The term “progressive supranuclear palsy” or “PSP” refers to a neurologic disorder of unknown origin that gradually destroys cells in many areas of the brain and the accumulation of abnormal aggregates of the microtubule-associated protein Tau, resulting in insoluble paired helical filaments, including the gradual deterioration of neurons and glial cells in the midbrain and frontal cortex that display insoluble helical filaments of Tau proteins as described e.g. in Shi et al., Nature 2021; 598:359-363.

PSP starts with a pre-symptomatic phase during which there is an increase in neuropathological abnormalities. Next, patients develop isolated symptoms that are suggestive of PSP (soPSP). In any of the methods described herein, PSP can be classic PSP-Richardson's syndrome (PSP-RS), PSP-Parkinsonism (PSP-P), PSP-corticobasal syndrome (PSP-CBS), PSP-progressive non-fluent aphasia (PSP-PNFA), or PSP-pure akinesia with gait freezing (PSP-PAGF) (Ling et al., J. Mov. Discord. 9(1):3-13, 2016). After onset, symptoms of PSP become rapidly and progressively worse. Subjects diagnosed with PSP may become severely disabled within five years and die within six years. Symptoms of PSP usually first appear at the age of 60 and worsen until death. People with PSP commonly die from pneumonia, choking or other complications caused by the loss of functional brain cells, resulting in loss of autonomic and motor function (e.g. the ability to swallow).

Signs and symptoms of PSP include movement, cognitive and psychiatric disorders. Voluntary movement can be impaired in PSP and include pseudobulbar palsy (i.e. inability to control facial movements), bradykinesia (i.e. slow or abnormal muscle movement), neck and trunk rigidity, impaired gait, impaired balance, posture instability and difficulty with speech and swallowing. The most obvious, outward sign of the disease is an inability to coordinate and move the eyes normally, resulting in a vertical gaze palsy. Cognitive impairments include loss of executive functions (e.g. attention control, inhibitory control, working memory, cognitive flexibility, reasoning, problem solving and planning) and diminished fluency. Associated psychiatric symptoms include depression, feelings of irritability, sadness or apathy, insomnia, fatigue and loss of energy. In some embodiments, a subject can be identified as having PSP using the MDS PSP Diagnostic Criteria (as described in, e.g., Hoglinger et al., Mov. Disord. 31:644-652, 2016).

In some embodiments, a subject can be identified as having an increased risk of developing PSP or identified as having PSP (e.g., any of the types of PSP described herein), e.g., at least in part, by detecting tau protein deposits (e.g., 4-repeat tau protein deposits), detecting of atrophy of the midbrain and/or superior cerebellar peduncles (e.g., using any of the imaging techniques described herein or known in the art, e.g., magnetic resonance imaging (MRI) or positron emission tomography (PET) scans), and/or detecting of hypometabolism in the frontal cortex, caudate, and/or thalamus in the subject (e.g., using any of the imaging techniques described herein or known in the art, e.g., MRI, CT scan, or PET scan).

In some embodiments, the present invention provides a pharmaceutical composition comprising a peptide comprising the amino acid sequence NAPVSIPQ (NAP peptide) and a pharmaceutically acceptable carrier, for use in a sex-specific treating or inhibiting the development or progression of the PSP in a subject. According to one embodiment, the subject is a female.

Therefore, in some embodiments, the present invention provides a pharmaceutical composition comprising a peptide comprising the amino acid sequence NAPVSIPQ (NAP peptide) and a pharmaceutically acceptable carrier for use in treating or inhibiting the development of progressive supranuclear palsy (PSP) in a female subject.

According to some embodiments, the female subject is 50 years old or more. According to some embodiments, the female subject is 55 years old or more. According to some embodiments, the female subject is 60 years old or more. According to some embodiments, the female subject is 65 years old or more. According to some embodiments, the female subject is 70, 75, 80, 85 or 90 years old or more.

According to some embodiments, the female subject is administered from 1 to 100 mg/day of the NAP peptide. According to some embodiments, the subject is administered from 1 to 70 mg/day of the NAP peptide. According to some embodiments, the female subject is administered from 2 to 65 mg/day, from 5 to 60 mg/day, of the NAP peptide. According to some embodiments, the female subject is administered from 10 to 50 mg/day, from 15 to 45 mg/day, from 20 to 40 mg/day or from 25 to 35 mg/day, or about 30 mg/day of the NAP peptide. According to some embodiments, the female subject is administered from 40 to 80 mg/day, from 45 to 75 mg/day, from 50 to 70 mg/day, from 55 to 65 mg/day or about 60 mg/day of the NAP peptide. According to any one of the above embodiments, the administration may be performed in one dose or split into several doses, e.g. 2, 3, or 4 doses.

In some embodiments, the present invention provides a pharmaceutical composition comprising a peptide comprising the amino acid sequence NAPVSIPQ (NAP peptide) and a pharmaceutically acceptable carrier for use in treating or inhibiting the development of progressive supranuclear palsy (PSP) in a male subject. According to some embodiments, the male subject is 50 years old or more. According to some embodiments, the male subject is 55 years old or more. According to some embodiments, the male subject is 60 years old or more. According to some embodiments, the male subject is 65 years old or more. According to some embodiments, the male subject is 70, 75, 80, 85 or 90 years old or more. According to some embodiments, the male subject is administered from 1 to 50 mg/day of the NAP peptide. According to some embodiments, the subject is administered from 1 to 45 mg/day of the NAP peptide. According to some embodiments, the male subject is administered from 2 to 40 mg/day, or from 3 to 35 mg/day, of the NAP peptide. According to some embodiments, the male subject is administered from 1 to 30, from 5 to 35 mg/day, from 10 to 40 mg/day or from 10 to 35 mg/day of the NAP peptide. According to some embodiments, the male subject is administered from 10 to 50 mg/day, from 15 to 45 mg/day, from 20 to 40 or from 25 to 35 mg/day, or about 30 mg/day of the NAP peptide. According to some embodiments, the male subject is administered from 1 to 15 mg/day, from 2 to 10 mg/day, from 3 to 10 mg/day, from 1 to 5 mg/day or from 2 to 4 mg/day of the NAP peptide. According to any one of the above embodiments, the administration may be performed in one dose or split in several doses, e.g., 2, 3, or 4 doses.

According to some embodiments, the efficacy of the treatment may be assessed by measuring any known symptom or parameter associated with the disease. According to some embodiments, the treatment results in improving one or more of said symptoms of parameters. Thus, according to some embodiments, the treatment comprises an improvement in at least one of the following symptoms associated with PSP: behavior, bulbar, dysarthria, ocular motor, limb motor and gait. According to other embodiments, the treatment comprises an improvement in the score of Geriatric Depression Scale and/or Clinical Global Impression of Disease Severity. According to other embodiments, the treatment comprises an improvement in the score of the Schwab and England Activities of Daily Living scale (SEADL).

The term “improved” when referring to symptoms or parameters of any one of the diseases described in the application may be quantitative and qualitative as known in the art. The improvement is measured/assessed in comparison to a subject that does not receive the treatment, i.e. a control. Thus, the term may have the meaning of improvement of at least 5%, 10%, 15%, 20%, 25%, 30%, 35%, 40%, 50%, 60%, 70%, 80%, 90%, or 95% in symptoms and/or parameters for a treated individual or a patient population compared to an individual or patient population not receiving the therapeutic agent. According to a further embodiment, the improvement is at least 1.5, at least 2, at least 2.5 at least 3, at least 5 or at least 10 folds compared to an individual or patient population not receiving the therapeutic agent.

### Schizophrenia

According to other embodiments, the disease is schizophrenia. The term “schizophrenia refers to a mental disorder as known in the art and described e.g. in DSM-IV-TR.

In some embodiments, the present invention provides a pharmaceutical composition comprising a peptide comprising the amino acid sequence NAPVSIPQ (NAP peptide) and a pharmaceutically acceptable carrier for use in a sex-specific treating schizophrenia in a subject.

According to one embodiment, the subject is female. Therefore, in some embodiments, the present invention provides a pharmaceutical composition comprising a peptide comprising the amino acid sequence NAPVSIPQ (NAP peptide) and a pharmaceutically acceptable carrier for use in treating schizophrenia in a female subject. According to some embodiments, the female subject is administered from 1 to 20 mg/day of the NAP peptide. According to some embodiments, the subject is administered from 1 to 15 mg/day of the NAP peptide. According to some embodiments, the female subject is administered from 2 to 10 mg/day, from 3 to 8 mg/day, of the NAP peptide. According to some embodiments, the female subject is administered from 1 to 4, from 1 to 3, or from 2 to 4 mg/day of NAP peptide. According to some embodiments, the female subject is administered about 1, about 1.5, about 2, about 2.5, about 3, about 3.5 or about 4 mg/day of NAP peptide. According to some embodiments, the female subject is administered about 1, about 1.5, about 2, about 2.5, about 3, about 3.5 or about 4 mg of NAP peptide b.i.d. According to some embodiments, the female subject is administered from 4 to 5 mg/day or about 5 mg/day of the NAP peptide. According to any one of the above embodiments, the administration may be performed in one dose or split in several doses, e.g. 2, 3, or 4 doses.

In some embodiments, the present invention provides a pharmaceutical composition comprising a peptide comprising the amino acid sequence NAPVSIPQ (NAP peptide) and a pharmaceutically acceptable carrier for use in treating schizophrenia in a male subject. According to some embodiments, the male subject is administered from 10 to 80 mg/day of the NAP peptide. According to some embodiments, the male subject is administered from 15 to 75 mg/day of the NAP peptide. According to some embodiments, the male subject is administered from 15 to 70 mg/day, from 20 to 60 mg/day, of the NAP peptide. According to some embodiments, the male subject is administered with from 20 to 40, or from 25 to 35 mg/day. According to some embodiments, the male subject is administered with from 25 to 60 mg/day or from 30 to 50 mg/day of the NAP peptide. According to some embodiments, the male subject is administered with from 40 to 80 mg/day, from 50 to 70 mg/day, or from 55 to 65 of the NAP peptide. According to some embodiments, the male subject is administered with about 30, about 40, about 50, or about 60 mg/day of the NAP peptide. According to any one of the above embodiments, the administration may be performed in one dose or split in several doses, e.g. 2, 3, or 4 doses.

Amnestic Mild Cognitive Impairment (aMCI)

According to other embodiments, the disease is amnestic mild cognitive impairment (aMCI). The term “amnestic mild cognitive impairment” refers to a mild cognitive impairment with memory loss as the predominant symptom and is frequently seen as a prodromal stage of Alzheimer's disease. For example, a person may start to forget important information that he or she would previously have recalled easily, such as appointments, conversations or recent events.

In some embodiments, the present invention provides a pharmaceutical composition comprising a peptide comprising the amino acid sequence NAPVSIPQ (NAP peptide) and a pharmaceutically acceptable carrier for use in a sex-specific treating aMCI in a subject.

According to one embodiment, the subject is female. Therefore, in some embodiments, the present invention provides a pharmaceutical composition comprising a peptide comprising the amino acid sequence NAPVSIPQ (NAP peptide) and a pharmaceutically acceptable carrier for use in treating aMCI in a female subject. According to some embodiments, the female subject is administered from 20 to 80 mg/day of the NAP peptide, once or twice daily. According to some embodiments, the female subject is administered from 25 to 70 mg/day or from 30 to 70 mg/day of the NAP peptide. According to some embodiments, the female subject is administered from 35 to 75 mg/day, from 40 to 70 mg/day, or from 40 to 65 of the NAP peptide. According to some embodiments, the female subject is administered from 40 to 80 mg/day, from 50 to 70 mg/day, or from 55 to 65 of the NAP peptide. According to some embodiments, the female subject is administered with about 30, about 35, about 40, about 45, about 50, about 55, or about 60 mg/day of the NAP peptide.

In some embodiments, the present invention provides a pharmaceutical composition comprising a peptide comprising the amino acid sequence NAPVSIPQ (NAP peptide) and a pharmaceutically acceptable carrier for use in treating aMCI in a male subject. According to some embodiments, the male subject is administered from 10 to 80 mg/day of the NAP peptide. According to some embodiments, the male subject is administered from 15 to 75 mg/day of the NAP peptide. According to some embodiments, the male subject is administered from 15 to 70 mg/day, from 20 to 60 mg/day, of the NAP peptide. According to some embodiments, the male subject is administered with from 20 to 40, or from 25 to 35 mg/day. According to some embodiments, the male subject is administered from 25 to 70 mg/day or from 30 to 70 mg/day of the NAP peptide. According to some embodiments, the male subject is administered from 35 to 75 mg/day, from 40 to 70 mg/day, or from 40 to 65 of the NAP peptide. According to some embodiments, the male subject is administered from 40 to 80 mg/day, from 50 to 70 mg/day, or from 55 to 65 of the NAP peptide. According to some embodiments, the male subject is administered with about 30, about 35, about 40, about 45, about 50, about 55, or about 60. According to any one of the above embodiments, the administration may be performed in one dose or split in several doses, e.g. 2, 3, or 4 doses.

### Patients at Risk for Alzheimer's Disease

In some embodiments, the present invention provides a pharmaceutical composition comprising a peptide comprising the amino acid sequence NAPVSIPQ (NAP peptide) and a pharmaceutically acceptable carrier for use in the sex-specific prevention of Alzheimer's disease.

### Alzheimer's Disease

According to other embodiments, the disease is Alzheimer's disease. The term “Alzheimer's disease” refers to a progressive neurologic disease of the brain that leads to the irreversible loss of neurons and dementia. The clinical hallmarks of Alzheimer's disease are progressive impairment in memory, judgment, decision making, orientation to physical surroundings, and language.

In some embodiments, the present invention provides a pharmaceutical composition comprising a peptide comprising the amino acid sequence NAPVSIPQ (NAP peptide) and a pharmaceutically acceptable carrier for use in a sex-specific treating Alzheimer's disease in a subject.

According to one embodiment, the subject is female. Therefore, in some embodiments, the present invention provides a pharmaceutical composition comprising a peptide comprising the amino acid sequence NAPVSIPQ (NAP peptide) and a pharmaceutically acceptable carrier for use in treating Alzheimer's disease in a female subject. According to some embodiments, the female subject is administered from 1 to 80 mg/day of the NAP peptide. According to some embodiments, the subject is administered from 1 to 70 mg/day of the NAP peptide. According to some embodiments, the female subject is administered from 2 to 65 mg/day, from 5 to 60 mg/day, of the NAP peptide. According to some embodiments, the female subject is administered from 10 to 50 mg/day, from 15 to 45 mg/day, from 20 to 40 mg/day or from 25 to 35 mg/day of the NAP peptide or about 30 35 mg/day of the NAP peptide. According to some embodiments, the female subject is administered from 40 to 80 mg/day, from 45 to 75 mg/day, from 50 to 70 mg/day, from 55 to 65 mg/day or about 60 mg/day of the NAP peptide. According to some embodiments, the female subject is administered from 1 to 20 mg/day of the NAP peptide. According to some embodiments, the subject is administered from 1 to 15 mg/day of the NAP peptide. According to some embodiments, the female subject is administered from 2 to 10 mg/day, from 3 to 8 mg/day, of the NAP peptide. According to some embodiments, the female subject is administered from 4 to 5 mg/day or about 5 mg/day of the NAP peptide. According to any one of the above embodiments, the administration may be performed in one dose or split in several doses, e.g. 2, 3, or 4 doses. According to one embodiment, the dose of the NAP peptide is about 5 mg once daily or about 15 mg twice daily.

In some embodiments, the present invention provides a pharmaceutical composition comprising a peptide comprising the amino acid sequence NAPVSIPQ (NAP peptide) and a pharmaceutically acceptable carrier for use in treating Alzheimer's disease in a male subject. According to some embodiments, the male subject is administered from 5 to 30 mg/day of the NAP peptide. According to some embodiments, the male subject is administered from 1 to 80 mg/day of the NAP peptide. According to some embodiments, the subject is administered from 1 to 70 mg/day of the NAP peptide. According to some embodiments, the female subject is administered from 2 to 65 mg/day, from 5 to 60 mg/day, of the NAP peptide. According to some embodiments, the female subject is administered from 10 to 50 mg/day, from 15 to 45 mg/day, from 20 to 40 mg/day or from 25 to 35 mg/day of the NAP peptide. According to some embodiments, the female subject is administered from 40 to 80 mg/day, from 45 to 75 mg/day, from 50 to 70 mg/day, from 55 to 65 mg/day or about 60 mg/day of the NAP peptide. According to some embodiments, the female subject is administered from 1 to 20 mg/day of the NAP peptide. According to some embodiments, the subject is administered from 1 to 15 mg/day of the NAP peptide. According to some embodiments, the female subject is administered from 2 to 10 mg/day, from 3 to 8 mg/day, of the NAP peptide. According to some embodiments, the female subject is administered from 4 to 5 mg/day or about 5 mg/day of the NAP peptide.

According to any one of the above embodiments, the Alzheimer's disease is at the early stage of the disease. According to some embodiments, the subject is at the prodromal stage of the disease. Thus, in some embodiments, the present invention provides a pharmaceutical composition comprising a peptide comprising the amino acid sequence NAPVSIPQ (NAP peptide) and a pharmaceutically acceptable carrier for use in sex-specific prevention of Alzheimer's disease in a male subject. According to some embodiments, the subject is pre- or post-Alzheimer's disease onset and is characterized by the presence in a biological sample of least one of the following biomarkers (i) of aberrant P53 protein; (ii) P-tau217; (iii) P-tau231 and (iii) P-tau181and (iv) change in ADNP expression as described e.g. in US2010303785. The term “biological sample” encompasses a variety of sample types obtained from an organism that may be used in a diagnostic or monitoring assay. The term encompasses blood, serum and other liquid samples of biological origin, solid tissue samples, such as a biopsy specimen, or tissue cultures or cells derived therefrom and the progeny thereof. The term specifically encompasses a clinical sample and includes serum, plasma, urine, biological fluids including aqueous humour and vitreous for eyes samples, and tissue samples. The term also encompasses samples that have been manipulated in any way after procurement, such as treatment with reagents, solubilization, or enrichment for certain components.

The biological sample could be a blood or serum sample. The treatment according to some embodiments is commenced before the clinical signs of Alzheimer's disease appear. According to some embodiments, the treatment comprises an improvement in the score of Geriatric Depression Scale of a subject suffering from Alzheimer's disease

According to some embodiments, the disease or disorder is autism. According to some embodiments, the disease or disorder is muscle disease associated with or mediated by aberrant functionality of ADNP. In some embodiments, the present invention provides a pharmaceutical composition comprising NAP and a pharmaceutically acceptable carrier for use in treating the disease or disorder in a female subject. According to some embodiments, the female subject is administered from 1 to 80 mg/day of the NAP peptide. According to some embodiments, the subject is administered from 1 to 70 mg/day of the NAP peptide. According to some embodiments, the female subject is administered from 2 to 65 mg/day, from 5 to 60 mg/day, of the NAP peptide. According to some embodiments, the female subject is administered from 10 to 50 mg/day, from 15 to 45 mg/day, from 20 to 40 mg/day or from 25 to 35 mg/day of the NAP peptide. According to some embodiments, the female subject is administered from 40 to 80 mg/day, from 45 to 75 mg/day, from 50 to 70 mg/day, from 55 to 65 mg/day or about 60 mg/day of the NAP peptide. According to some embodiments, the female subject is administered from 1 to 20 mg/day of the NAP peptide. According to some embodiments, the subject is administered from 1 to 15 mg/day of the NAP peptide. According to some embodiments, the female subject is administered from 2 to 10 mg/day, from 3 to 8 mg/day, of the NAP peptide. According to some embodiments, the female subject is administered from 4 to 5 mg/day or about 5 mg/day of the NAP peptide. In some embodiments, the present invention provides a pharmaceutical composition comprising a peptide comprising the amino acid sequence NAPVSIPQ (NAP peptide) and a pharmaceutically acceptable carrier for use in treating the disease or disorder in a male subject. According to some embodiments, the male subject is administered from 1 to 80 mg/day of the NAP peptide. According to some embodiments, the male subject is administered from 1 to 70 mg/day of the NAP peptide. According to some embodiments, the male subject is administered from 2 to 65 mg/day, from 5 to 60 mg/day, of the NAP peptide. According to some embodiments, the male subject is administered from 10 to 50 mg/day, from 15 to 45 mg/day, from 20 to 40 mg/day or from 25 to 35 mg/day of the NAP peptide. According to some embodiments, the male subject is administered from 40 to 80 mg/day, from 45 to 75 mg/day, from 50 to 70 mg/day, from 55 to 65 mg/day or about 60 mg/day of the NAP peptide. According to some embodiments, the female subject is administered from 1 to 20 mg/day of the NAP peptide. According to some embodiments, the male subject is administered from 1 to 15 mg/day of the NAP peptide. According to some embodiments, the male subject is administered from 2 to 10 mg/day, from 3 to 8 mg/day, of the NAP peptide. According to some embodiments, the male subject is administered from 4 to 5 mg/day or about 5 mg/day of the NAP peptide According to any one of the above embodiments, the administration may be performed in one dose or split in several doses, e.g. 2, 3, or 4 doses.

According to yet another aspect, the present invention provides a pharmaceutical composition comprising a NAP peptide and a pharmaceutically acceptable carrier for use in ameliorating and/or preventing side effects of an anti-amyloid-β therapy, wherein the therapy comprises anti-amyloid-β antibodies.

As used herein, the term “anti-amyloid-β antibodies” refer to antibodies that bind specifically to amyloid-β and are used for treatment of Alzheimer's disease. According to some embodiments, the anti-amyloid-β therapy is selected from aducanumab, bupineuzuman, gantenerumab, gantenerumab, lecanemab, and solanezumab. According to some embodiments, the anti-amyloid-β therapy is aducanumab.

According to some embodiments, the composition is administered in a sex-specific mode. According to some embodiments, the subject is a female. According to some embodiments, the composition is administered to a female subject. According to some embodiments, the female subject is administered from 1 to 80 mg/day of the NAP peptide. According to some embodiments, the subject is administered from 1 to 70 mg/day of the NAP peptide. According to some embodiments, the female subject is administered from 2 to 65 mg/day, from 5 to 60 mg/day, of the NAP peptide. According to some embodiments, the female subject is administered from 10 to 50 mg/day, from 15 to 45 mg/day, from 20 to 40 mg/day or from 25 to 35 mg/day of the NAP peptide. According to some embodiments, the female subject is administered from 40 to 80 mg/day, from 45 to 75 mg/day, from 50 to 70 mg/day, from 55 to 65 mg/day or about 60 mg/day of the NAP peptide. According to some embodiments, the female subject is administered from 1 to 20 mg/day of the NAP peptide. According to some embodiments, the subject is administered from 1 to 15 mg/day of the NAP peptide. According to some embodiments, the female subject is administered from 2 to 10 mg/day, from 3 to 8 mg/day, of the NAP peptide. According to some embodiments, the female subject is administered from 4 to 5 mg/day or about 5 mg/day of the NAP peptide.

According to some embodiments, the subject is a male. In some embodiments, the pharmaceutical composition comprising the NAP peptide is administered to a male subject. According to some embodiments, the male subject is administered from 1 to 80 mg/day of the NAP peptide. According to some embodiments, the male subject is administered from 1 to 70 mg/day of the NAP peptide. According to some embodiments, the male subject is administered from 2 to 65 mg/day, from 5 to 60 mg/day, of the NAP peptide. According to some embodiments, the male subject is administered from 10 to 50 mg/day, from 15 to 45 mg/day, from 20 to 40 mg/day or from 25 to 35 mg/day of the NAP peptide. According to some embodiments, the male subject is administered from 40 to 80 mg/day, from 45 to 75 mg/day, from 50 to 70 mg/day, from 55 to 65 mg/day or about 60 mg/day of the NAP peptide. According to some embodiments, the male subject is administered from 1 to 20 mg/day of the NAP peptide. According to some embodiments, the male subject is administered from 1 to 15 mg/day of the NAP peptide. According to some embodiments, the male subject is administered from 2 to 10 mg/day, from 3 to 8 mg/day, of the NAP peptide. According to some embodiments, the male subject is administered from 4 to 5 mg/day or about 5 mg/day of the NAP peptide According to any one of the above embodiments, the administration may be performed in one dose or split in several doses, e.g. 2, 3, or 4 doses.

According to any one of the above embodiments, the use comprises administering the pharmaceutical composition for at least one day before starting anti-amyloid-β therapy. According to any one of the above embodiments, the use comprises administering the pharmaceutical composition for at least 2, 3, 4, 5, 6 or 7 days before starting anti-amyloid-β therapy. According to any one of the above embodiments, the use comprises administering the pharmaceutical composition for at least 2, 3, 4, 5, 6, 7 or 8 weeks or 2, 3, 4, 5, 6, 7, 8, 9, 10, 11 or 12 months before starting anti-amyloid-P therapy.

According to some embodiments, wherein the use comprises administering the pharmaceutical composition in combination with the anti-amyloid-P therapy.

According to yet another aspect, the present invention provides a method of treating or inhibiting the development or progression of a disease or disorder associated with an aberrant functionality of activity-dependent neuroprotective protein (ADNP) in a subject, wherein the treatment is gender specific.

According to yet another aspect, the present invention provides a method of treating or inhibiting the development or progression of progressive supranuclear palsy (PSP) in a female subject, the method comprises administering a peptide comprising the amino acid sequence NAPVSIPQ (SEQ ID NO: 1).

According to yet another aspect, the present invention provides a method of treating or inhibiting the development or progression of progressive supranuclear palsy (PSP) in a male subject, the method comprises administering from 1 to 50 mg/day of a peptide comprising the amino acid sequence NAPVSIPQ (SEQ ID NO: 1).

According to yet another aspect, the present invention provides a method of treating schizophrenia in a male subject, the method comprises administering from 10 to 80 mg/day of a peptide comprising the amino acid sequence NAPVSIPQ (SEQ ID NO: 1).

According to yet another aspect, the present invention provides a method of treating or inhibiting the development or progression of aMCI in a male subject, the method comprises administering from 20 to 80 mg/day of a peptide comprising the amino acid sequence NAPVSIPQ (SEQ ID NO: 1).

According to yet another aspect, the present invention provides a method of treating or inhibiting the development or progression of aMCI in a female subject, the method comprises administering from 35 to 80 mg/day of a peptide comprising the amino acid sequence NAPVSIPQ (SEQ ID NO: 1).

According to yet another aspect, the present invention provides a method of preventing the development of Alzheimer's disease, preferably in a sex-specific manner/mode.

According to yet another aspect, the present invention provides a method of treating schizophrenia in a female subject, the method comprises administering from 1 to 4 mg/day of a peptide comprising the amino acid sequence NAPVSIPQ (SEQ ID NO: 1).

According to yet another aspect, the present invention provides use of a peptide comprising the amino acid sequence NAPVSIPQ (SEQ ID NO: 1) for preparing a medicament for sex-specific treating or inhibiting the development or progression of a disease or disorder associated with an aberrant functionality of activity-dependent neuroprotective protein (ADNP) in a subject.

According to yet another aspect, the present invention provides use of a peptide comprising the amino acid sequence NAPVSIPQ (SEQ ID NO: 1) for preparing a medicament for treating or inhibiting the development or progression of progressive supranuclear palsy (PSP) in a female subject.

According to yet another aspect, the present invention provides use of a peptide comprising the amino acid sequence NAPVSIPQ (SEQ ID NO: 1) for preparing a medicament for treating schizophrenia in a male subject, wherein the treating comprises administering medicament comprise administering from 15 to 80 mg/day of the peptide.

According to any one of the embodiments and aspects of the present invention, the treatment using NAP may be combined with any treatment commonly used for the corresponding disease.

The invention will now be illustrated by the following non-limiting Examples.

Having now generally described the invention, the same will be more readily understood through reference to the following examples, which are provided by way of illustration and are not intended to be limiting of the present invention.

The terms “a,” “an,” and “the” are used herein interchangeably and mean one or more.

The term “and/or” is used to indicate one or both stated cases may occur, for example A and/or B includes, (A and B) and (A or B).

The term “or,” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. Therefore, the term “comprising the amino acid sequence” encompasses the term “consisting of the amino acid sequence” and may be replaced by it. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

### EXAMPLES

### Example 1. NAP for Sex-Specific Treatment of Progressive Supranuclear Palsy

### Methods

In this example we used results of a clinical trial performed as described in Boxer et al. Shortly, in a double blind, parallel group, phase 2/3 trial, participants were randomly assigned with permuted blocks in a 1:1 ratio to davunetide (NAP, having amino acid sequence NAPVSIPQ set forth as SEQ ID NO: 1, 30 mg twice daily, intranasally) or placebo for 52 weeks at 48 centers in Australia, Canada, France, Germany, the UK, and the USA. Participants met the modified Neuroprotection and Natural History in Parkinson Plus Syndrome study criteria for PSP. Primary endpoints were the change from baseline in PSP Rating Scale (PSPRS, 28 items, 6 categories, 0=normal, 100=most disability) and Schwab and England Activities of Daily Living scale (SEADL, 11-point rating scale, 0=vegetative, 100%/=completely independent, 10% increments) at up to 52 weeks. Data collection was at week 0, 6, 13, 26, 39 and 52. All participants and study personnel were masked to treatment assignment. Analysis was by intention to treat. The trial is registered with Clinicaltrials.gov, number NCT01110720. 313 participants were randomly assigned to davunetide (n=157) or to placebo (n=156), and 241 (77%) completed the study (118 and 156 in the davunetide and placebo groups, respectively). A secondary outcome was brain ventricular volume as measured with boundary shift integral analysis of T1-weighted magnetic resonance imaging (MRI) scans, mostly collected at week 0 and 52. At the same time, selected patients were also subjected to lumbar puncture and assessment of Tau content and Tau hyperphosphorylation in the cerebrospinal fluid (CSF). Boxer et al concluded that Davunetide is not an effective treatment for PSP and that clinical trials of disease-modifying treatment are feasible in patients with PSP and should be pursued with other promising tau-directed treatments.

To further understand disease progression, sex/age/baseline differences and drug influence, we re-evaluated study results, separating males and females, and assessing the primary and secondary endpoints. In this respect, we further assessed individually the 6 disability domains of the PSPRS, namely: daily activities (by history, seven items with a total maximum score of 24), behavior (mentation, emotional, interference with activity of daily living, maximum score of 16), bulbar (speech and swallowing, maximal score of 8), ocular motor (eye and eyelid activity, 16 points), limb motor (fine motor skills including tremor, 16 points) and gait/midline (gross motor skills, 20 points) https://www.physio-pedia.com/Progressive_Supranuclear_Palsy_Rating_Scale_(PSP-RS).

Statistical considerations: To include the ventricle volumetric measures, the cohort was composed of female and male PSP patients treated with davunetide or placebo (sample sizes for sex-treatment groups at 52 weeks of treatment: 48-55), outcome variables included ventricular volume (will be referred to as volume), PSPRS and SEADL assessed at study initiation and follow up to 52 weeks after twice daily treatment.

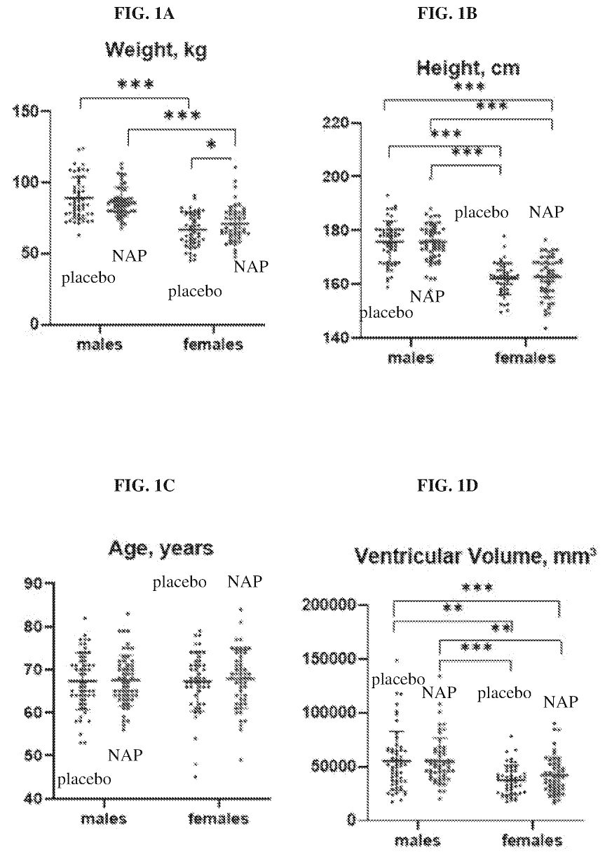

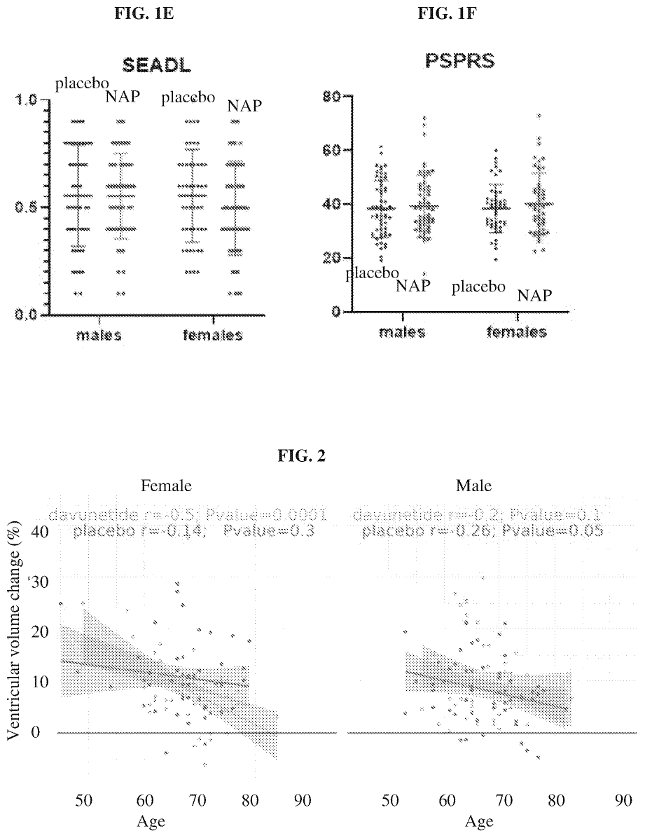

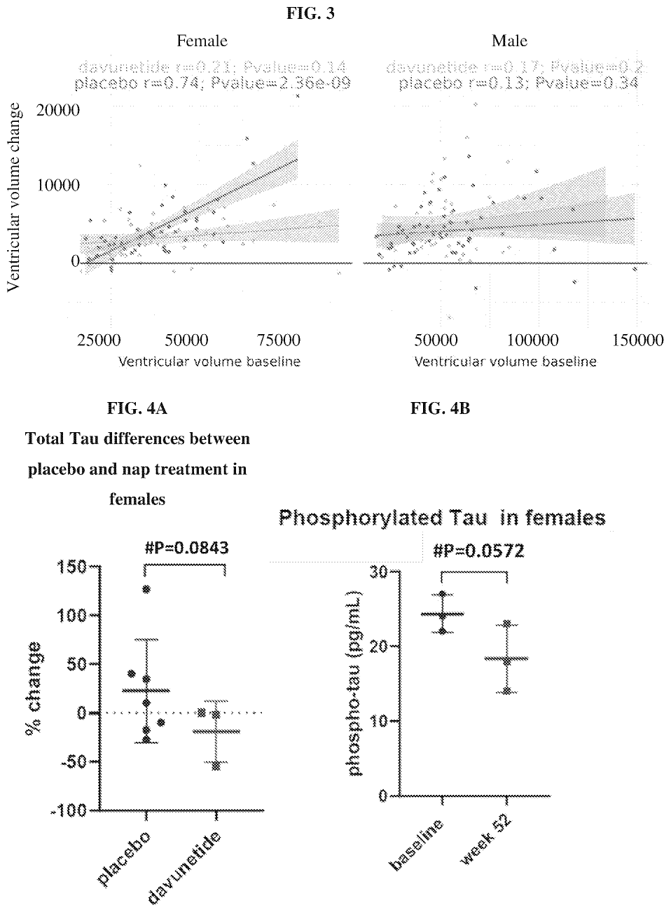

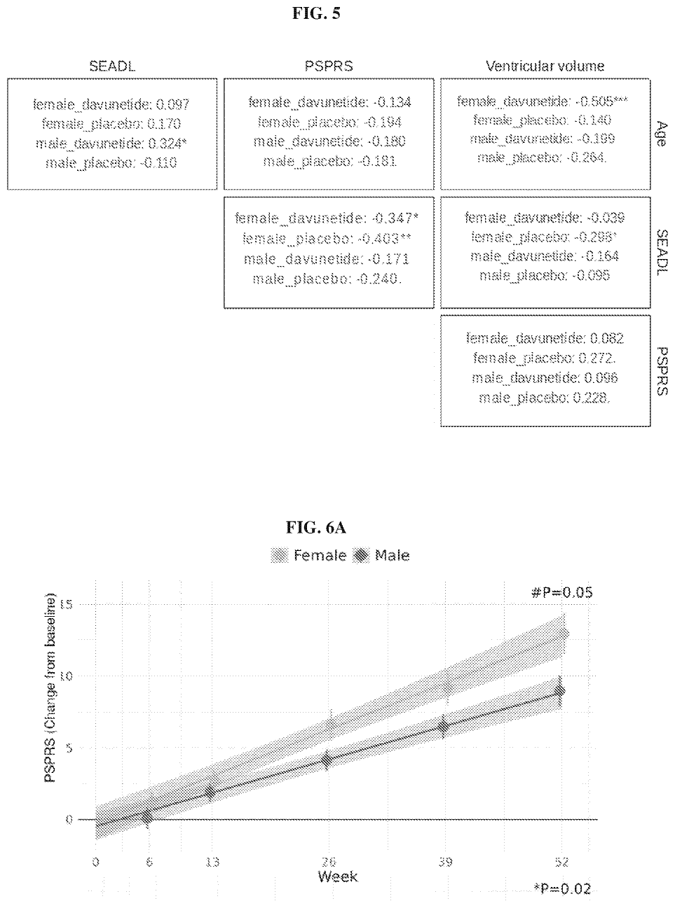

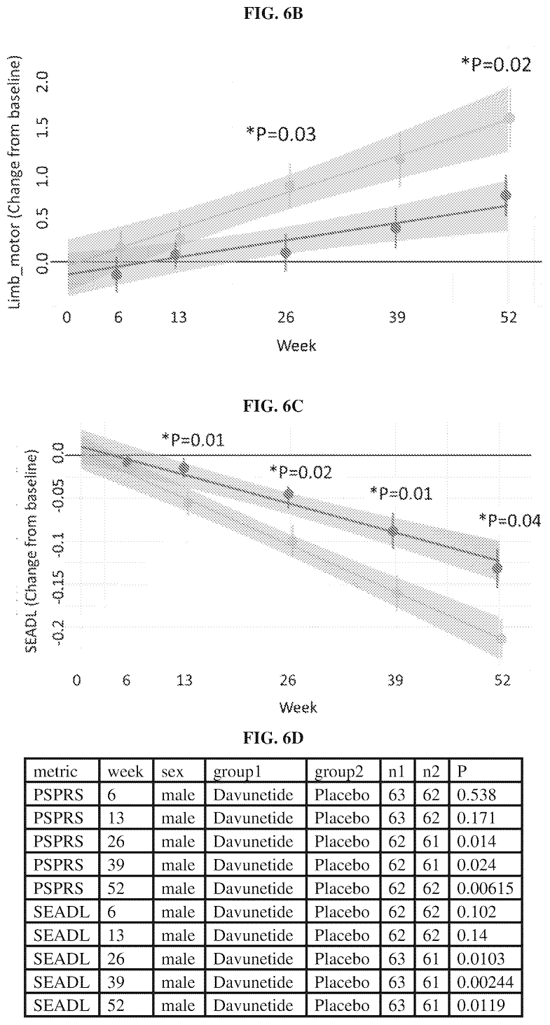

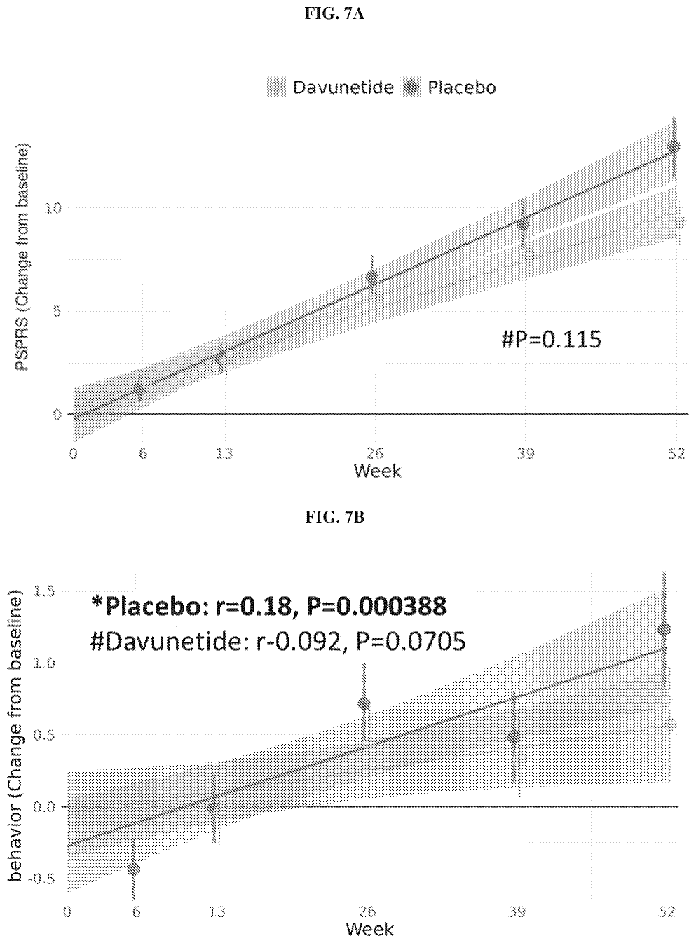

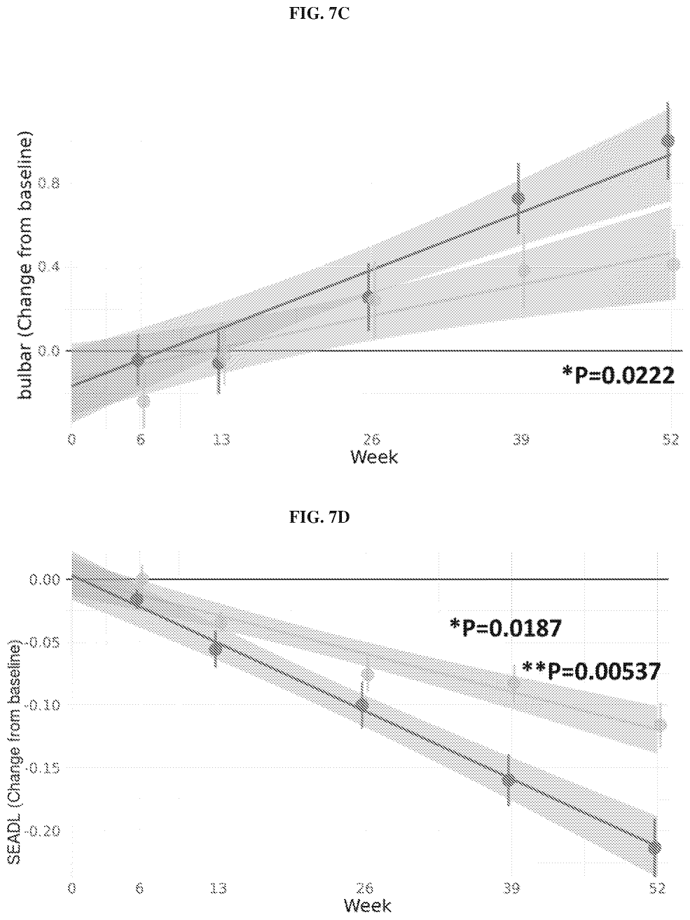

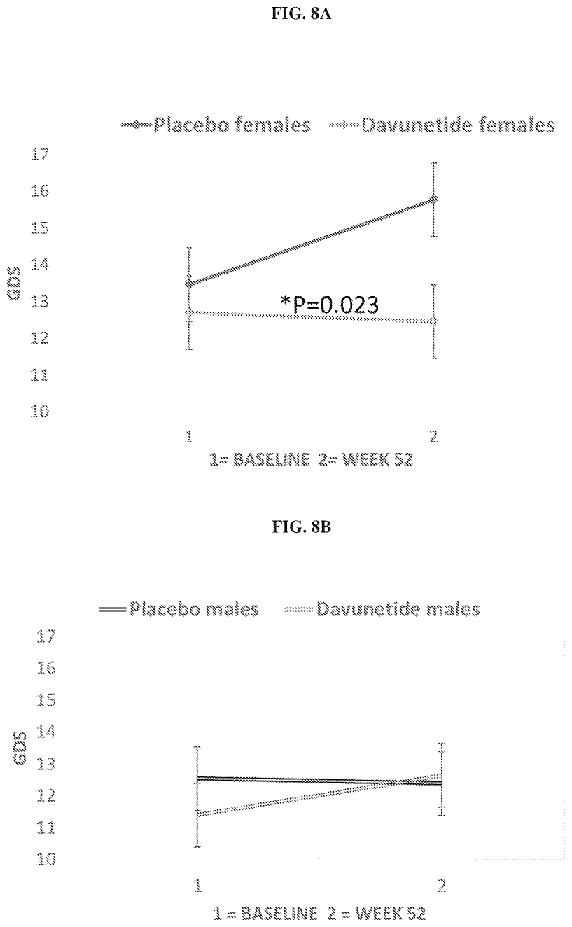

Data analysis was performed using Graph Pad Prism 7.0 software (see FIGS. 1 and 4). Other statistical tests and visualizations were made in R version 4.2.1 (2022-06-23) with the base library, ggplot2 and ggplot2 extension packages. Correlation plots statistics (FIGS. 2, 3 and 5) were computed using the standard Pearson method. The volume change (from FIG. 3) is the difference between the baseline and week-52 measurements. PSPRS, SEADL and ventricular volume in figures (FIG. 5) are represented by differences between the baseline and week-52 measurements, as a percentage of the baseline measurement. FIGS. 6 and 7 give the absolute changes overtime, due to the non-normal distribution of most parameters, statistical significance was measured using the Wilcoxon test. Only extreme values (defined as 3*IQR deviation from the penultimate top/bottom quantiles), were removed from the final calculations. There were only 11 observations that were considered extreme and their influence on our results was insignificant. FIG. 8 representing the geriatric depression scale measure (GDS) was prepared using excel.

### Results

### Significant Baseline Sex Differences

FIG. 1 reveals that at the baseline, the tested population differed by weight and height, with obvious higher male values. Furthermore, on average, the randomized female davunetide group showed significantly increased weight compared to the placebo group. Age-wise, there were no differences.

Concentrating on brain structure and as expected from previous evaluations, MRI measures of ventricular volumes were larger in males at baseline compared to the tested females, suggesting that sex separation in the analysis of the results is required for a neuroprotective drug trial, regardless of the original design.

No differences were seen in PSP Rating Scale (PSPRS) and Schwab and England ADL (SEADL) scale baseline measures. However, closer inspection (Student's t test, one tail) comparing sex-specific davunetide-treated with the placebo groups showed a trending for increased average ventricular volume and PSPRS measures (P=0.0716 and P=0.0934, respectively) and decreased SEADL measures (P=0.0934) in females only, potentially indicating a disadvantageous starting point for the davunetide-treated female group, vs. the placebo group (FIG. 1D-F)

### Davunetide Treatment Provides Significant Neuroprotection Against Age/Brain Ventricular Volume-Dependent Increases in PSP Woman Patients

Given the significant sex and age differences in ventricular volumes, with davunetide target being neuroprotection, we have initially focused on percent of baseline ventricular volume changes with age vs. davunetide protective effects. While the placebo group did not show a difference in the percentage of ventricular volume increase during the trial period (52 weeks) across the age groups, namely, there was a constant deterioration, davunetide treatment showed a significant neuroprotective effect in the older woman patients (FIG. 2). No differences between the davunetide and the placebo group were observed in men (FIG. 2). Therefore, while the alteration in the ventricular volume change in davunetide-treated men across the age groups was comparable to that of the placebo group during the trial period (52 weeks), a significant difference in the change of ventricular volume in comparison to the placebo group was observed for women treated with davunetide indicating for its neuroprotective effect in the older woman patients, (FIG. 2). Further focusing on age, the most beneficial davunetide effects were discovered in women older than 60 years of age. Thus, we discovered significant age/decreased ventricular volumes correlations in davunetide-treated females (r=−0.5, P=0.0001) (FIG. 2), while male patients showed no effects. Furthermore, FIG. 3, separating the study population by sex and baseline ventricular volume measures, showed a dramatic increase correlating with the baseline ventricular volumes (r=0.74; P=2.36⁻⁹) in the placebo group, with a complete protection by davunetide treatment, in females. Contrary to females, the male cohorts showed no dependence on baseline ventricular volumetric measures, and no drug effects (FIG. 3).

Similar results were seen at the entire population level (Student's t test, two tails, *P-0.04, and −#P=0.056; Wilcoxon test, for female, davunetide protection). Exploratory results looking at very few individuals indicated that the woman cohort also showed trends in total Tau and phosphorylated Tau reduction in the cerebrospinal fluid (CSF) of the davunetide-treated patients compared to placebo (FIG. 4), experiments were performed as detailed in Rojas et al (Neurology 2018; 90(4): e273-e281).

Davunetide Neuroprotection is Significantly Correlated with the Study Primary Endpoints: SEADL and PSPRS

FIG. 5 depicts correlation plots of percent change compared to baseline at 52-week davunetide vs. placebo treatment results including the study primary endpoints, PSPRS and SEADL, the secondary end point of brain ventricular volume change as well as age.

The male disease course seemed to differ from the female also in the fact that the only significant correlation seen males was with the baseline age and the SEADL change in the davunetide group (FIG. 5). 11401 Furthermore, and in agreement with the results above (FIG. 2), ventricular volume significantly correlated with age only in females (r=−0.505***). This female-specific davunetide protection on ventricular volume was correlated with the primary endpoints of the trial, SEADL, r=−0.298*, and PSPRS (almost significant, r=0.272, only for the placebo groups), indicative of davunetide neuroprotection, translated into clinical protection. Additionally, the SEADL and the PSPRS percent change from baseline to 52 treatment weeks were further correlated in the female group (r=−0.403**, placebo and r=−0.347*, davunetide).

### Significant Sex Differences and Protection Against SEADL Deterioration by Davunetide Treatment in Women

Given the observed sex differences, in ventricular volumes, with a dramatic volume increases correlating with the baseline levels in placebo-treated-females only and davunetide protection (FIG. 3), we looked at clinical disease progression in females and males separately. FIG. 6 shows changes in PSPRS over time, namely 6, 13, 26, 39 and 52 weeks after study initiation in the placebo groups, defining disease progression. Significantly accelerated disease progression was observed in females, almost reaching significance at 52 weeks (#P=0.05, FIG. 6A). The PSPRS measures disability across 28 items in six domains: daily activities (by history), behavior, bulbar, ocular motor, limb motor and gait/midline. As such, we also looked at the 6 different domains, revealing significant differences between males and females in limb motor activity (fine motor skills), with *P=0.03 and 0.02, at weeks 26 and 52, respectively (FIG. 6B).

These findings indicate, sex-dependent disease progression. FIG. 6C further shows a dramatic difference in SEADL deterioration between males and females reaching significance already at 13 weeks (**P=0.01), and continuing thereafter.

When looking at efficacy in males, the davunetide treated group showed significant increase in PSPRS and decrease in SEADL, from week 26 on compared to the placebo group, mimicking the more rapid diseases progression in females (FIG. 6D).

In contrast, in females, davunetide treatment resulted in PSPRS measures that trended toward significant improvement at 52 weeks, (FIG. 7A). A transient adverse effect on oculomotor activities on week 6 (P=0.0127) was detected. However, no adverse effects were noted for all other PSPRS domains. Interestingly, while in the placebo group, a significant deterioration was discovered for the female mental behavior (***r=0.18, P=0.000388, measuring disorientation, bradyphrenia including slowness of thought processes and inattentiveness, emotional incontinence and grasping behavior affecting activities of daily living), with davunetide treatment, disease progression was almost halted, which is indicative of disease modification (#r-0.092, P=0.0705, FIG. 7B). Furthermore, a significant protective effect on the bulbar domain of the PSPRS was discovered. Davunetide protected against dysarthria, difficulty speaking caused by brain damage, which results in an inability to control the muscles used in speech and dysphagia, difficulty swallowing, at week 52 of treatment (FIG. 7C, *P=0.0222). Additionally, the limb motor skills showed to deteriorate faster in females vs. males (FIG. 6B) and were partially protected by davunetide treatment in females (#P=0.0838). Lastly, davunetide efficacy in females was extraordinary in SEADL (change from baseline) showing a significant protective effect starting at week 39, and continuing on 52 (FIG. 7D, *P=0.0187 and **P=0.00537, respectively). Thus, we have discovered a female-specific target engagement and protection against increases in ventricular volume, coupled with a significant protection on bulbar functional activities and no significant deterioration of mental activities within the PSPRS, correlated with of activities of daily living (SEADL).

Further, we have reevaluated additional secondary endpoint CGIC (Clinical Global Impression of Change). Exploratory end points included CGIds (Clinical Global Impression of Disease Severity), GDS (Geriatric Depression scale) and RBANS (Repeatable Battery for the Assessment of Neuropsychological Disease Severity).

Calculating 52 week change as fraction proportional to baseline measures and expanding the analysis first to the additional secondary endpoint, CGIC, indicated significant correlation with SEADL (r=−0.306*, −0.304* for the female davunetide-treated and placebo groups, respectively and −0.307* for the male placebo). CGIds correlated most significantly with SEADL (r=−0.470*** and −0.408**, female davunetide and placebo, respectively, and −0.301** for the male, placebo). CGIds also highly correlated with the patient age for the female placebo group and the male davunetide group (r=−0.483*** and −0.421**, respectively). Age also correlated well with CGIC for the male davunetide group (−0.331*). In agreement with the results above, CGIds also correlated with ventricular volume only for the female placebo group (0.405**). The GDS score showed correlations only in the female placebo group, almost significantly with ventricular volume (r=0.253.) and significantly with CGIds and SEADL (r=0.323*, −0.310*, respectively). In this respect, Wilcoxon testing indicated that the GDS score (measuring geriatric depression) improved significantly in the davunetide-treated females compared to placebo, at week 52 (*P=0.023), while there was a highly significant sex-difference in the placebo groups (**P=0.007) (FIG. 8). Contrarily, the CGIds deteriorated only in the davunetide-treated males (*P=0.013) with the sex-dependent davunetide-treated group difference (P=*0.02).

Other exploratory endpoints showed additional significant correlations, for example, phonemic fluency correlated most significantly with CGIds only in the female placebo group (r=0.447**), in agreement with davunetide protection. In contrast, in males, phonemic fluency correlated mostly with letter number sequence (r=0.556*** davunetide, r=0.419** placebo) as well as with GDS and SEADL in the placebo group (r=0.306*, 0.281*, respectively). Letter number sequencing correlated with CGIC only in davunetide-treated males (r=0.288*). The color trails2 test, a neuropsychological assessment of executive function (Boxer et al.), trended toward correlation with CGIC in the female davunetide group (r=0.254.).

### Discussion

The surprising female-specific PSP-related neurodegeneration observed as increases in ventricular volumes (depending on the baseline starting point) in the current study. Thus, we discovered a sex-dependent differential vulnerability of ventricular volume measure, indicative of accelerated neurodegeneration in female PSP patients compared to males. We further discovered differential disease progression, in men and women, with women progressing faster in what seems to be fine motor deterioration. Furthermore, the PSP female patient brain reacts differently to davunetide treatment than the PSP male brain as seen by the age- and baseline volume-dependent manner, requiring neuroprotection, with davunetide protecting microtubule cytoskeletal interactions, brain structure and axonal transport, regulated by ADNP in a sex-dependent manner.

Falls within 1 year of disease onset, apathy and executive dysfunction are more frequent in PSP woman patients and time to attain unintelligible speech, severe dysphagia and cognitive impairment are earlier in females, in agreement with the SEADL correlation with brain ventricular volume in females and not in males, the male-female differences in the SEADL progression and in davunetide bulbar protection, in females, in the current study.

These findings emphasize the requirement for drug and dose selection, which is appropriate for the study population.

Regardless, the correlation of behavioral outcomes with ventricular volume protection is of high significance. A most recent review cited the progressive, dose-dependent ventricular enlargement in the brains of patients with early Alzheimer's disease who were treated with the FDA-approved drug aducanumab (Ayton S. Nature reviews Neurology 2022; 18(7): 383-384)—an anti-amyloid-β therapy. Our current findings imply that davunetide protects against this unwanted potential side effect of amyloid-targeting drugs as well as protect against disease-related ventricular volume loss, presenting a first of its kind drug with shown human efficacy in the select woman population suffering from PSP and beyond. Improvement in GDS=Geriatric Depression Scale in PSP suffering women indicates that the treatment may be efficient for the same indication in subjects suffering from geriatric depression, a major risk for Alzheimer's disease as well as Alzheimer's disease depression, considering the common mechanisms behind these diseases and further indicating treatment for depression in general, in a sex-dependent dosing regimen.

### Example 2. Prevention of Alzheimer's Disease Neurodeaeneration

Computational analysis by the eukaryotic linear motif (ELM) prediction tool was performed and several caspase cleavage sites were identified in ADNP including the following classes: cysteinyl aspartate specific proteases; The subtilisin-like proprotein convertases (PCSKs) mammalian subtilisin/kexin isozymes (SKIs); and paired basic amino acid cleaving system 4, SPC4.

Caspases represent key players in apoptosis, development and differentiation. Caspases recognize the respective substrates by specific cleavage motifs. There are five amino acids of the substrate around the caspase cleavage site, named (N- to C-terminal): P4, P3, P2, P1, P-1. The scissile bond between the essential aspartate at P1 and P-1, usually a small amino acid, is cleaved by caspase-3 and -7, whereas positions P4 to P-1 are important for substrate specificity and recognition.

We found that ADNP residues 734-738 contain the motif DDSDS which is a recognition motif for caspase-3 and caspase-7. Cleavage of the caspase substrates results in characteristic morphological features of apoptotic cell death, including membrane blebbing, pyknotic nuclei, cell rounding, and formation of apoptotic vesicles. Thus, activated caspase-3, a major enzyme in the apoptotic pathway, is often used as a marker for apoptotic cells. The length of the ADNP protein after the caspase cleavage is 737aa. Interestingly, one of the most prevalent autism/intellectual disability causing de novo mutations in ADNP in p.Arg730*, closely located near the caspase cleavage site. Furthermore the recurrent somatic ADNP frameshift mutation p.Arg730Thrfs*4, which is one of the pathogenic mutations in ADNP that is correlated to aging/Alzheimer's disease, truncates ADNP length to a protein of 734aa.

Subtilisin-Like Proprotein Convertases (PCSKs) Mammalian Subtilisin/Kexin Isozymes (SKIs) are Expressed Extensively in Mammalian Neural and Endocrine Cells and Play a Major Role in the Proteolytic Processing of Both Neuropeptide and Peptide Hormone Precursors. The Members of the Subtilisin-Like Family are Proprotein Convertases that Process Latent Precursor Proteins into Biologically Active Products. PCSK1 (Proprotein Convertase 1, NEC1) and PCSK2 (Proprotein Convertase 2, NEC2) are Type I Proinsulin-Processing Enzymes Important in Regulating Insulin Biosynthesis. These Enzymes are Also Known to Cleave Proopiomelanocortin, Prorenin, Proenkephalin, Prodynorphin, Prosomatostatin and Progastrin. 11581 We determined that ADNP residues 367-371 KQLLP include the cleavage motif recognized by the members of the subtilisin-like family. The length of ADNP after cleavage in this site is 367aa. Interestingly, the length of ADNP after the truncating mutation p.Ile359Thrfs*8 is 367aa, found in the post mortem Alzheimer's brain.

PACE4 (paired basic amino acid cleaving system 4. SPC4) is a calcium-dependent serine endoprotease that can cleave precursor protein at paired basic amino acid processing sites (e.g. p.Lys20*, found in the postmortem Alzheimer's brain). Its substrates include transforming growth factor beta-related proteins, proalbumin, and von Willebrand factor and assorted neuropeptides. Furthermore, several paired basic amino acids are found in ADNP.

### Discussion

Previous studies have shown that NAP, a short active motif within ADNP, protects against activated caspase 3 associated apoptosis. Our findings, together with previous observations suggest that ADNP cleavage by caspase 3 may be deleterious at two levels: 1) enhancing DNA damage, and 2) reducing ADNP-Tau-microtubule interactions, resulting in tauopathy and followed or paralleled by apoptosis. These findings implicate ADNP as part of the apoptotic pathways in neuronal cells.

Also interesting and related are the findings of caspase 3-dependent proteolytic cleavage of Tau causes neurofibrillary tangles and results in cognitive impairment during normal aging. This is coupled with the finding of plasma P-tau217 levels increasing during the early preclinical stages of Alzheimer's disease when insoluble tau aggregates are not yet detectable by tau-positron emission tomography (PET), presenting an early biomarker. However, an even earlier biomarker is suggested in an unfolded conformational variant of P53, apparent at least 6 years prior to disease onset. Phosphorylated Tau and modified P53 in prodromal Alzheimer's disease are also associated with ADNP found to be the only protein decreasing in Alzheimer's disease patients' serum samples and with ADNP serum levels correlating with intelligence, in cognitively intact healthy elderly. Importantly, ADNP indirectly interacts with sirtuin 1 (SIRT1) at the chromatin and microtubule/Tau levels as well as regulates Forkhead box O3 (FOXO3), two important genes associated with healthy aging.

Taken together, our studies suggest ADNP directed therapy in susceptible individuals exhibiting one or more of the modified P53 biomarker, P-tau217 and Phosphorylated Tau, identified in body fluids and by imaging. A suggested therapy would be nasal NAP (davunetide) administration with previous human experience and cognitive score protection/enhancement in amnestic mild cognitive impairment (aMCI) patients.

Interestingly, when specifically analyzing ADNP (the parent protein of the NAP peptide) in RNA sequencing results (Wang et al., J Clin Invest. 2022; 132(2):e149904), we now discovered a statistically significant increase in its expression only in the Alzheimer's disease samples vs. control (about 70-80 samples per group), and only in the temporal cortex. Specifically, the FDR=False Discovery Rate for the temporal cortices indicated a very high significance (0.0008) for a higher ADNP expression in the Alzheimer's disease samples compared to controls.

### Example 3. Sex-Specific Treatment of Amnestic Mild Cognitive Impairment

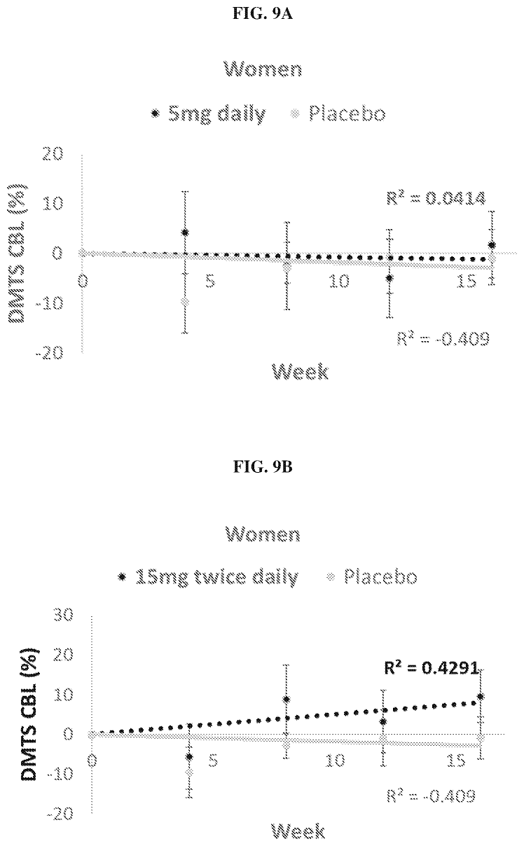

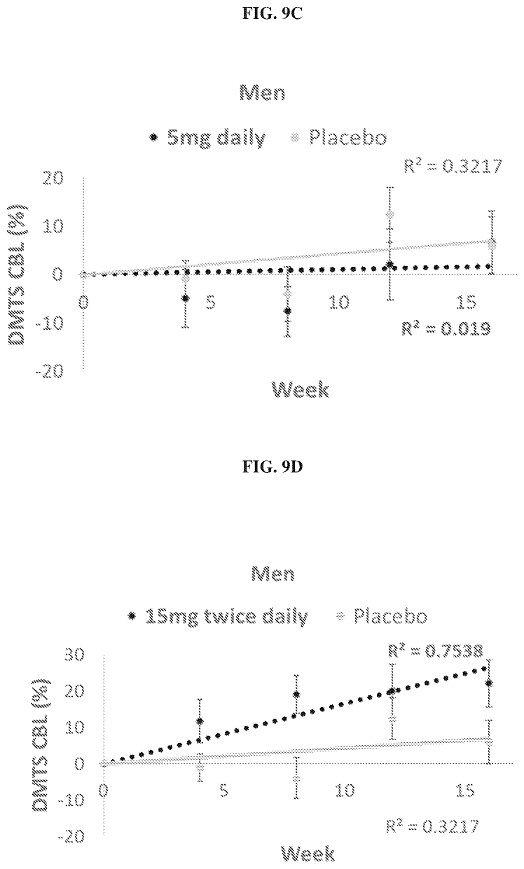

In line with the above-described Examples, we analyzed results of the aMCI clinical trial (Gozes et al., Curr Alzheimer Res. 2009; 6(5):455-60; Morimoto et al., Dement Geriatr Cogn Disord. 2013; 35(5-6):325-36.) which tested the effect of NAP peptide on subjects suffering from an amnestic mild cognitive impairment (aMCI) (a precursor to Alzheimer's disease (AD). When re-analyzing the results, we separated men and women and found a surprisingly sex-related differences when testing the efficacy by the CANTAB Delayed Match-to-Sample DMTS test. DMTS is a test of delayed and recognition memory using simultaneous and delayed visual matching to sample. In this task, the subject is shown a complex visual pattern on a screen and after a delay (simultaneous, 0, 4 or 12 s), 4 patterns are displayed. The subject's task is to select the pattern that matches the original sample, here depicted after a 12 s interval. Thus, subjects suffering from aMCI were treated with either 5 mg daily or 15 mg twice daily (BID) by intranasal davunetide administration (1 or three puffs of the same strength davunetide solution). Treatment was for 12 weeks and testing was at baseline and every four weeks, including also 4 weeks after cessation of treatment. When analyzing the data, we separated here men and women for the first time as outlined below. 76 women were divided into three groups (25 or 26 women in each group with the placebo group divided approximately into half each receiving the same placebo (formulation without drug) volume, and the two other groups receiving NAP peptide (davunetide, AL-108). In parallel, a group of 68 men similarly divided was tested. Notably, all previous analyses did not separate men and women. Now, analyzing men and women separately, our new surprising results show a correlation of improvement with time of r²=0.4291 for women and r²=0.7538 for men treated with 15 mg davunetide twice daily, contrasting an r² of −0.409 for women in the placebo group and r² of 0.3217 for men (FIG. 9, indicating sex-related differences). Furthermore, Student's t tests at all testing time points indicated a significant difference in test week 8 and trending on week 4 and 16, in men only, further contrasting the PSP results and emphasizing the surprising sexual/disease differences. We show that treatment of males with 30 mg/day NAP peptide provided a statistically significant effect, which was not observed or anticipated in the previous publications. We further conclude that much higher dose are required, especially in females. As such doses of from 60 to 80 mg/day of NAP are suggested.

### Example 4. Sex-Specific Treatment of Schizophrenia

Javitt et al., (Schizophrenia research 2012; 136(1-3): 25-31) showed that NAP (davunetide) provides protection in the UCSD Performance-based Skills Assessment (UPSA) in schizophrenia patients. Briefly, sixty-three subjects with schizophrenia received davunetide at one of two different doses (5, 30 mg/day) or placebo for 12 weeks in a multicenter, double-blind, parallel-group randomized clinical trial. The MATRICS Consensus Cognitive Battery (MCCB) assessed cognitive effects. The UCSD Performance-based Skills Assessment (UPSA) with maximal score of 100 and the Schizophrenia Cognition Rating Scale (SCoRS) assessed functional capacity. Subjects continued their current antipsychotic treatment during the trial. For UPSA, there was a significant main effect of treatment across study arms (p=0.048). Between-group effect size (d) values were 0.74 and 0.48, favoring the 5 and 30 mg doses, respectively. Given that the data mixed men and women, it was assumed that no differences were observed between the sexes.

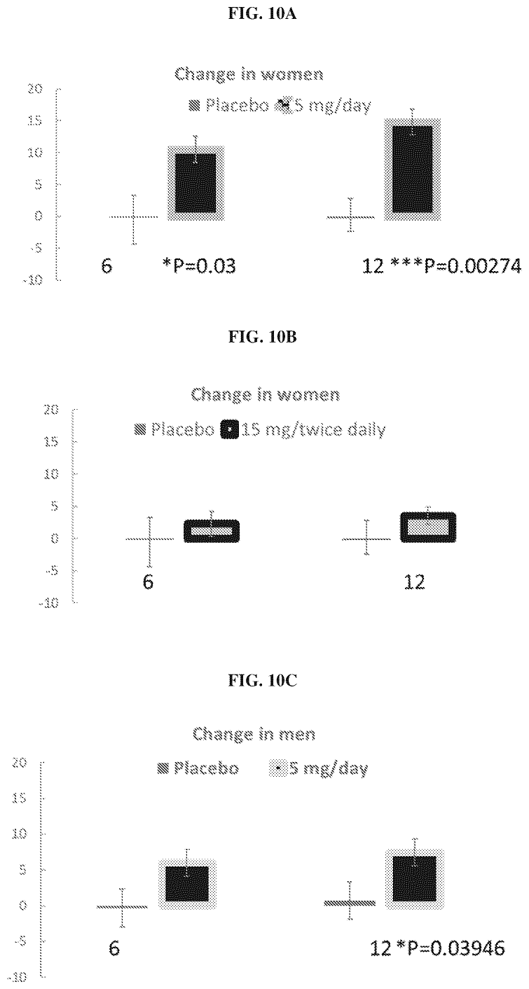

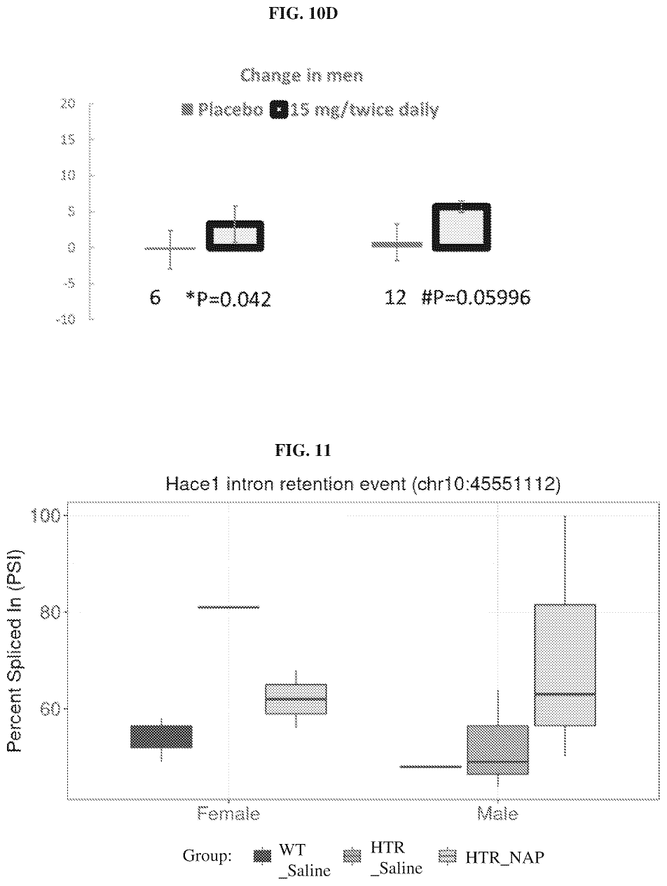

Considering the results obtained for PSP and the involvement of ADNP in both diseases, we analyzed the data separately for males and females with 7-16 subjects per group. We found a surprising significant difference at the 5 mg daily-treated sex groups (*P=0.0276). Thus, treated women had a mean of 14.9 (15% improvement after 12 weeks), while men were trending toward improvement with an average of 7.4 points (FIG. 10A and. FIG. 10C, respectively). A further surprise included the unexpected finding that increasing the dose in women, resulted in a significantly lower effect on UPSA, namely, at week 12 the 5 mg/day treated women were significantly better than the 15 mg/twice daily treated women (FIG. 10 A vs. B, ***P=0.002). All in all, the 5 mg daily dose was beneficial for women as detected after 6 and 12 weeks of treatment. While the same low dose was effective in men as well (FIG. 10C) especially after 6 weeks of treatment, the 15 mg/twice-daily treatment showed surprising significant results only in men, treated for 12 weeks (FIG. 10 D). This is contrary to Javitt's statements that 15 mg/bid is not significantly effective on UPSA.

Returning to females, the surprising results suggest lower than 5 mg daily davunetide doses will be efficacious, strongly claiming sex-dose differences. Considering the bell-shape response, we conclude that a dose below 5 mg/day, as low as 1-4 mg/day provides the maximum effect in schizophrenia treatment in women.

Linear regression analysis in comparing age (42-56 years of age) to UPSA changes, showed the most interesting positive correlation with the most efficacious dose in women, namely, 5 mg/daily for 12 weeks, (R²=0.2356), with surprising apparently increased positive drug effects in the older women, claiming age/efficacy effect not seen before.

### Example 5

In this experiment we analyzed hippocampal RNA sequencing of Adnp mutated (called HTR (Tyr)) mice, exhibiting tauopathy (Karmon et al., Biol Psychiatry. 2022; 92(1):81-95). In this model, gait deficits were accentuated in females, and ameliorated by NAP treatment. We discovered a surprising mirror imaging in NAP peptide (davunetide) brain (hippocampus) action in the treated mice as a dependence on mice sex. Here we show, for the first time (FIG. 11) a sex-specific opposite expression as a consequence of NAP treatment of Hace1 (HECT Domain And Ankyrin Repeat Containing E3 Ubiquitin Protein Ligase 1). Hace1 is associated with ataxia and Huntington disease and is also involved in depression and Alzheimer's disease. NAP protection (FIG. 11) further attests to sex-dependent treatments of the diseases associated or mediated with Hace1.

Although the present invention has been described herein above by way of preferred embodiments thereof, it can be modified, without departing from the spirit and nature of the subject invention as defined in the appended claims.

1-35. (canceled)

36. A method for a sex-specific treating or inhibiting of the development of a disease or disorder associated with an aberrant functionality of activity-dependent neuroprotective protein (ADNP) in a subject in need thereof comprising administering to said subject a peptide comprising the amino acid sequence NAPVSIPQ (SEQ ID NO: 1; NAP peptide) and a pharmaceutically acceptable carrier.

37. The method according to claim 36, wherein the disease or disorder is selected from a tauopathy, neurodegenerative disease, neurodevelopmental disease and mental disease.

38. The method according to claim 36, wherein the disease or disorder is selected from progressive supranuclear palsy (PSP), schizophrenia, amnestic mild cognitive impairment (aMCI), Alzheimer's disease, ADNP syndrome, autism and muscle disease.

39. The method according to claim 38, wherein the disease is progressive supranuclear palsy (PSP) and the method comprises treating or inhibiting the development of PSP in a female subject.

40. The method according to claim 39, wherein the treating or preventing is characterized in at least one of:
(i) the treating or preventing the development of PSP comprises preventing or inhibiting an increase in the size of brain ventricles;
(ii) the treating comprises improvement in at least one of the following symptoms associated with PSP: behavior, bulbar, dysarthria, ocular motor, limb motor and gait, parameters of the Schwab and England Activities of Daily Living scale (SEADL) and/or improvement of the parameters of Geriatric Depression Scale and Clinical Global Impression of Disease Severity;
(iii) the female subject is 60 years old or more;
(iv) the pharmaceutical composition is administered intranasally;
(v) use comprises administering from 1 to 80 mg/day or of the NAP peptide or from 40 to 80 mg/day of the NAP peptide.

41. The method according to claim 38, wherein the disease is schizophrenia and the use comprises treating schizophrenia in a male subject, wherein the method comprises administering to said subject from 10 to 80 mg/day of the NAP peptide.

42. The method according to claim 38, wherein the disease is schizophrenia and the method comprises treating schizophrenia in a female subject by administering to said subject from 1 to 4 mg/day of the NAP peptide.

43. The method according to claim 38, wherein the disease is amnestic mild cognitive impairment (aMCI) and the method comprises treating amnestic aMCI in a male subject by administering to said subject from 10 to 80 mg/day of the NAP peptide.

44. The method according to claim 38, wherein the disease is amnestic mild cognitive impairment (aMCI) and the use comprises treating amnestic aMCI in a female subject, wherein the use comprises administering to said subject from 35 to 80 mg/day of the NAP peptide.

45. The method according to claim 38, wherein the method comprises sex-specific treatment or prevention of Alzheimer's disease.

46. The method according to claim 45, characterized by at least one of:
(i) the subject is at the prodromal stage of the disease;
(ii) the Alzheimer's disease is characterized by the presence in a biological sample of least one of the followings: (i) of aberrant P53 protein; (ii) P-tau217; (iii) P-tau231 and (iii) P-tau181 and (iv) change in ADNP expression; or
(iii) the method comprises treating a female subject by administering from 1 to 80 mg/day, from 1 to 10 mg/day, from 20 to 40 mg/day or from 40 to 80 mg/day of the NAP peptide; or
(iv) the method comprises treating a male subject and wherein the use comprises administering from 1 to 80 mg/day, from 1 to 10 mg/day, from 20 to 40 mg/day or from 40 to 80 mg/day of the NAP.

47. The method according to claim 45, comprising improving in Geriatric Depression Scale parameters.

48. The method according to claim 36, comprising intranasal administration of the peptide.

49. The method according to claim 36, comprising inhibiting neurodegeneration in the subject.

50. A method for ameliorating and/or preventing side effects of an anti-amyloid-β therapy in a subject in need thereof, the method comprises administering to said subject a peptide comprising the amino acid sequence NAPVSIPQ (SEQ ID NO: 1; NAP peptide) wherein the therapy comprises anti-amyloid-β antibodies.

51. The method according to claim 50, wherein the peptide is administered in a sex-specific mode.

52. The method according to claim 50, characterized by at least one of:
(i) wherein the peptide is administered to a female subject;
(ii) wherein the peptide is administered to a male subject;
(iii) the method comprises administering the peptide at least one day before starting anti-amyloid-β therapy; or
(iv) the method comprises administering the pharmaceutical composition in combination with the anti-amyloid-β therapy.

53. The method according to claim 50, comprising administering to a female subject from 1 to 70 mg/day or from 50 to 70 mg/day of the NAP peptide or administering to a to a male subject from 1 to 70, from 1 to 50 or from 50 to 70 mg/day of the NAP.

54. The method according to 52, wherein the anti-amyloid-β therapy comprises aducanumab or lecanemab.


SEQUENCE LISTING (49 sequences)

SEQ ID NO: 1 - protein, 8 aa, synthetic construct
  REGION 1..8: note = Synthetic polypeptide amino acid sequence
NAPVSIPQ

SEQ ID NO: 2 - protein, 18 aa, synthetic construct
  REGION 1..18: note = Synthetic polypeptide amino acid sequence
SVRLGLGGNAPVSIPQQS

SEQ ID NO: 3 - protein, 15 aa, synthetic construct
  REGION 1..15: note = Synthetic polypeptide amino acid sequence
LGLGGNAPVSIPQQS

SEQ ID NO: 4 - protein, 13 aa, synthetic construct
  REGION 1..13: note = Synthetic polypeptide amino acid sequence
LGGNAPVSIPQQS

SEQ ID NO: 5 - protein, 10 aa, synthetic construct
  REGION 1..10: note = Synthetic polypeptide amino acid sequence
GGNAPVSIPQ

SEQ ID NO: 6 - protein, 9 aa, synthetic construct
  REGION 1..9: note = Synthetic polypeptide amino acid sequence
  MOD_RES 1: note = ACETYLATION
NAPVSKIPQ

SEQ ID NO: 7 - protein, 9 aa, synthetic construct
  REGION 1..9: note = Synthetic polypeptide amino acid sequence
NAPVSKIPQ

SEQ ID NO: 8 - protein, 9 aa, synthetic construct
  REGION 1..9: note = Synthetic polypeptide amino acid sequence
NAPBSAIPQ

SEQ ID NO: 9 - protein, 9 aa, synthetic construct
  REGION 1..9: note = Synthetic polypeptide amino acid sequence
NAPVSRIPQ

SEQ ID NO: 10 - protein, 9 aa, synthetic construct
  REGION 1..9: note = Synthetic polypeptide amino acid sequence
NAPVTRIPQ

SEQ ID NO: 11 - protein, 9 aa, synthetic construct
  REGION 1..9: note = Synthetic polypeptide amino acid sequence
NAPVAAAAQ

SEQ ID NO: 12 - protein, 8 aa, synthetic construct
  REGION 1..8: note = Synthetic polypeptide amino acid sequence
  REGION 1..8: note = MISC_FEATURE - d-amino acid
NAPVSIPQ

SEQ ID NO: 13 - protein, 8 aa, synthetic construct
  REGION 1..8: note = Synthetic polypeptide amino acid sequence
AAPVSIPQ

SEQ ID NO: 14 - protein, 8 aa, synthetic construct
  REGION 1..8: note = Synthetic polypeptide amino acid sequence
NAAVSIPQ

SEQ ID NO: 15 - protein, 8 aa, synthetic construct
  REGION 1..8: note = Synthetic polypeptide amino acid sequence
NAPASIPQ

SEQ ID NO: 16 - protein, 8 aa, synthetic construct
  REGION 1..8: note = Synthetic polypeptide amino acid sequence
NAPVAIPQ

SEQ ID NO: 17 - protein, 8 aa, synthetic construct
  REGION 1..8: note = Synthetic polypeptide amino acid sequence
NAPVSAPQ

SEQ ID NO: 18 - protein, 8 aa, synthetic construct
  REGION 1..8: note = Synthetic polypeptide amino acid sequence
NAPVSIAQ

SEQ ID NO: 19 - protein, 8 aa, synthetic construct
  REGION 1..8: note = Synthetic polypeptide amino acid sequence
NAPVSIPA

SEQ ID NO: 20 - protein, 4 aa, synthetic construct
  REGION 1..4: note = Synthetic polypeptide amino acid sequence
SKIP

SEQ ID NO: 21 - protein, 4 aa, synthetic construct
  REGION 1..4: note = Synthetic polypeptide amino acid sequence
  MOD_RES 1: note = ACETYLATION
  MOD_RES 4: note = AMIDATION
SKIP

SEQ ID NO: 22 - skipped

SEQ ID NO: 23 - protein, 9 aa, synthetic construct
  REGION 1..9: note = Synthetic polypeptide amino acid sequence
SALLRSIPA

SEQ ID NO: 24 - protein, 14 aa, synthetic construct
  REGION 1..14: note = Synthetic polypeptide amino acid sequence
VAGGGSALLRSIPA

SEQ ID NO: 25 - protein, 19 aa, synthetic construct
  REGION 1..19: note = Synthetic polypeptide amino acid sequence
VEEGIVLGGGSALLRSIPA

SEQ ID NO: 26 - protein, 14 aa, synthetic construct
  REGION 1..14: note = Synthetic polypeptide amino acid sequence
VLGGGCALLRCIPA

SEQ ID NO: 27 - protein, 8 aa, synthetic construct
  REGION 1..8: note = Synthetic polypeptide amino acid sequence
ALLRSIPA

SEQ ID NO: 28 - protein, 10 aa, synthetic construct
  REGION 1..10: note = Synthetic polypeptide amino acid sequence
GSALLRSIPA

SEQ ID NO: 29 - protein, 6 aa, synthetic construct
  REGION 1..6: note = Synthetic polypeptide amino acid sequence
SALLRS

SEQ ID NO: 30 - protein, 10 aa, synthetic construct
  REGION 1..10: note = Synthetic polypeptide amino acid sequence
GSALLRSIPA

SEQ ID NO: 31 - protein, 12 aa, synthetic construct
  REGION 1..12: note = Synthetic polypeptide amino acid sequence
VAGGGSALLRSI

SEQ ID NO: 32 - protein, 10 aa, synthetic construct
  REGION 1..10: note = Synthetic polypeptide amino acid sequence
VLGGGSALLR

SEQ ID NO: 33 - protein, 9 aa, synthetic construct
  REGION 1..9: note = Synthetic polypeptide amino acid sequence
VLGGGSALL

SEQ ID NO: 34 - protein, 8 aa, synthetic construct
  REGION 1..8: note = Synthetic polypeptide amino acid sequence
VAGGGSAL

SEQ ID NO: 35 - protein, 9 aa, synthetic construct
  REGION 1..9: note = Synthetic polypeptide amino acid sequence
  REGION 1..9: note = MISC_FEATURE - d-amino acid
SALLRSIPA

SEQ ID NO: 36 - protein, 9 aa, synthetic construct
  REGION 1..9: note = Synthetic polypeptide amino acid sequence
AALLRSIPA

SEQ ID NO: 37 - protein, 9 aa, synthetic construct
  REGION 1..9: note = Synthetic polypeptide amino acid sequence
SAALRSIPA

SEQ ID NO: 38 - protein, 9 aa, synthetic construct
  REGION 1..9: note = Synthetic polypeptide amino acid sequence
SALARSIPA

SEQ ID NO: 39 - protein, 9 aa, synthetic construct
  REGION 1..9: note = Synthetic polypeptide amino acid sequence
SALLASIPA

SEQ ID NO: 40 - protein, 9 aa, synthetic construct
  REGION 1..9: note = Synthetic polypeptide amino acid sequence
SALLRAIPA

SEQ ID NO: 41 - protein, 9 aa, synthetic construct
  REGION 1..9: note = Synthetic polypeptide amino acid sequence
SALLRSAPA

SEQ ID NO: 42 - protein, 9 aa, synthetic construct
  REGION 1..9: note = Synthetic polypeptide amino acid sequence
SALLRSIAA

SEQ ID NO: 43 - protein, 26 aa, synthetic construct
  REGION 1..26: note = Synthetic polypeptide amino acid sequence
SALLRSIPAPAGASRLLLLTGEIDLP

SEQ ID NO: 44 - protein, 14 aa, synthetic construct
  REGION 1..14: note = Synthetic polypeptide amino acid sequence
VLGGGSALLRSIPA

SEQ ID NO: 45 - protein, 13 aa, synthetic construct
  REGION 1..13: note = Synthetic polypeptide amino acid sequence
LGGGSALLRSIPA

SEQ ID NO: 46 - protein, 12 aa, synthetic construct
  REGION 1..12: note = Synthetic polypeptide amino acid sequence
GGGSALLRSIPA

SEQ ID NO: 47 - protein, 11 aa, synthetic construct
GGSALLRSIPA

SEQ ID NO: 48 - protein, 88 aa, synthetic construct
  VARIANT 1..40: note = any naturally occurring amino acid
  VARIANT 49..88: note = any naturally occurring amino acid
XXXXXXXXXXXXXXXXXXXXXXXXXXXXXXXXXXXXXXXXNAPVSIPQXXXXXXXXXXXXXXXXXXXXXXXXXXXXXXXXXXXXXXXX

SEQ ID NO: 49 - protein, 89 aa, synthetic construct
  REGION 1..89: note = Synthetic polypeptide amino acid sequence
  VARIANT 1..40: note = any naturally occurring amino acid
  VARIANT 50..89: note = any naturally occurring amino acid
XXXXXXXXXXXXXXXXXXXXXXXXXXXXXXXXXXXXXXXXSALLRSIPAXXXXXXXXXXXXXXXXXXXXXXXXXXXXXXXXXXXXXXXX